(19) United States
(12) Patent Application Publication
(10) Pub. No.: US 2022/0365078 A1
(43) Pub. Date: Nov. 17, 2022

(54) NANOSTRUCTURE-BASED SENSORS AND METHODS FOR DETECTING ANTIGENS AND ANTIBODIES

(71) Applicant: University of Pittsburgh - of the Commonwealth System of Higher Education, Pittsburgh, PA (US)

(72) Inventors: Alexander Star, Pittsburgh, PA (US); Michael R. Shurin, Wexford, PA (US); Sarah Elizabeth Wheeler, Pittsburgh, PA (US); Wenting Shao, Pittsburgh, PA (US)

(21) Appl. No.: 17/739,800

(22) Filed: May 9, 2022

Related U.S. Application Data
(60) Provisional application No. 63/185,647, filed on May 7, 2021.

(51) Int. Cl. G01N 33/543; G01N 27/414

(52) U.S. Cl. CPC G01N 33/5438; G01N 27/4145

(57) ABSTRACT

A method of detecting an analyte in a fluid sample includes exposing a sensor including a substrate and a sensor medium on the substrate to the fluid sample for a period of time. The sensor medium includes a plurality of nanostructures and one or more of at least one agent selected from the group consisting of an antibody, an antigen receptor or an antigen immobilized upon at least a portion of the plurality of nanostructures. The at least one agent is an antibody or an antigen receptor if the analyte is an antigen and is an antigen if the analyte is an antibody. An electrolyte liquid having a known ionic strength which is less than the fluid sample is added over the sensor medium subsequent to exposing the sensor to the fluid and a variable providing a measure of change in at least one property of the sensor medium which is dependent upon the presence of the analyte is measured in presence of the electrolyte liquid.

### CROSS-REFERENCE TO RELATED APPLICATIONS

This application claims benefit of U.S. Provisional Patent Application Ser. No. 63/185647, filed May 7, 2021, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Early diagnosis of infections such as SARS-CoV-2 and other potential endemic or pandemic infections is very important for facilitating proper containment procedures, and a rapid, sensitive antigen assay is a desirable step M curbing an epidemic or pandemic, Rapid detection of infection (for example, SARS-CoV-2 infection) is likewise important for reducing morbidity and mortality of diseases such as Coronavirus disease 2019 (COVID-19). The current methodology in assessing infection with SARS-CoV-2 primarily relies OD nucleic acid amplification tests (NAATs) which detect the genetic material from SARS-CoV-2, While NAAT-based tests demonstrate excellent sensitivity for detection of viral RNA, such tests are high-complexity tests requiring specialized equipment and training, and shortages have resulted in significant delays (for example, up to a week) in reporting of results in some areas and time. periods.

Testing for SARS-CoV-2 antigens would be a beneficial addition to NAAT. Antigen detection, in general, is relatively inexpensive, can have a short turnaround time, and is amenable to point-of-care diagnostic methodologies. Currently existing rapid antigen testing tools in both laboratories and clinics are mostly based on lateral flow immunoassay (LFA) platforms. LEA is inexpensive and easily mass-produced, making it advantageous for rapid in-field detection of SARS-CoV-2 antigens, However, the sensitivity of LFA is generally not high enough to accurately screen COVID-19 patients.

Among several potentially useful detection methods for viral protein detection, field-effect transistor (FET)-based biosensing devices are advantageous as they offer high sensitivity, small size, and label-free and real-time detection. Recently, graphene-based FETs have been applied for COVID-19 detection. One such system provides a label-free graphene-FET immunosensor that purportedly identifies and captures the SARS-CoV-2 spike protein S1 within 2 min with a limit of detection of 0.2 pM. The detection relies on the highly specific interaction between SARS-CoV-2 spike protein S1 and the SARS-CoV-2 spike SI subunit protein antibody (CSAb) or human angiotensin-converting enzyme 2 (ACE2)-functionalized graphene surface. Another recently developed system includes a SARS-CoV-2 viral detection platform with a graphene-based FET biosensing device functionalized with the anti-SARS-CoV-2 spike antibody. The reported COVID-19 FET sensor detects SARS-CoV-2 spike proteins in nasopharyngeal swabs without preprocessing of the samples and can detect SARS-CoV-2 when RNA is present at 2.4×10² copies/mL. Both graphene-based FET sensors showed promise for applications in COVID-19 diagnosis, albeit with lower sensitivity compared to NAAT but with a short detection time.

Although significant research has been applied and a number of systems, devices, and methods have been developed to enable early, rapid and/or facile detection of infectious pathogens and other antigens, further technological developments for detection remain desirable.

### SUMMARY

In one aspect, a method of detecting an analyte which is an antigen or an antibody in a fluid sample includes providing a sensor device including a sensor including a substrate and a sensor medium on the substrate (such that the fluid sample contacts the sensor medium). The sensor medium includes a plurality of nanostructures having an enriched semiconducting content and one or more of at least one agent selected from the group consisting of an antibody, an antigen receptor or an antigen immobilized upon at least a portion of the plurality of nanostructures. The sensor device further includes electronic circuitry including at least one measurement system in operative connection with the sensor to measure a variable providing a measure of change in at least one property of the sensor medium which is dependent upon the presence of the analyte. The at least one agent is an antibody or an antigen receptor if the analyte is an antigen, and the at least one agent is an antigen if the analyte is an antibody. The method further includes exposing the sensor to the fluid sample for a period of time, subsequent to exposing the sensor to the fluid sample, washing the sensor one or more times with a liquid of known ionic strength, and after washing the sensor, measuring an output of the sensor with the liquid of known ionic strength over or covering the sensor medium/sensor. The liquid having a known ionic strength may be chosen to have an ionic strength that is less than the fluid sample to increase sensitivity compared to output measured in the presence of the fluid sample. In a number of embodiments, the sensor is washed a plurality of times. Such washing may, for example, remove unbound species. In a number of embodiments, the liquid is a purified water, In a number of embodiments, the liquid electrolyte has resistivity greater than or equal to18. 2 MΩ·cm.

The plurality of nanostructures may, for example., have an enriched semiconducting content of at least 66%, 90%, 99% or 99.9%. In a number of embodiments, the measured variable is an electrical property change (for example, resistance or conductance). In a number of embodiments, the at least one agent is maintained or is immersed in in a liquid phase.

In a number of embodiments, the plurality of nanostructures include carbon nanostructures. The plurality of carbon nanostructures may, for example, include or be composed of single-walled carbon nanotubes.

In a number of embodiments, the at least one agent is an antibody and the analyte is an antigen. The antigen may, for example, be an antigen of a pathogen selected from the group of a viral pathogen and a bacterial pathogen. The pathogen may, for example, be SARS-CoV-2, HA, tuberculosis, syphilis, hepatitis (for example., hepatitis b or c), E. coli, Salmonella, Pseudomonas aeruginosa, Influenza, Staphylococcus aureus, or Streptococcus pyogenes, cytomegalovirus (CAM, or Epstein-Barr virus (EB V). In a number of embodiments, the pathogen is SARS-CoV-2. The antigen may, for example, be a spike antigen (SAg) or a nucleocapsid protein antigen (NAg) of SARS-CoV-2 and the antibody is anti-SARS-CoV-2 spike protein antibody (SAb) or anti-SARS-CoV-2 nucleocapsid protein antibody. In a number of embodiments, the antigen is a spike antigen (SAg) of SA RS-CoV-2 and the antibody in the anti-SAS-CoV-2 spike protein antibody.

Many other antigens, including non-pathogenic antigens may be detected by the sensors hereof, Such antigens include, for example, substance originating within the body (for example, hormones such, as cortisol) and substances originating outside of the body (for example, drugs/opioids such as fentanyl/norfentanyl).

The sensor may, for example, be incorporated within a field effect transistor circuit or a chemiresistor circuit of the electronic circuitry. In a number of embodiments, the sensor is incorporated within a field effect transistor circuit and the liquid functions as a liquid gate.

The sensor device may, for example, include a plurality of the sensors. In a number of embodiments, the sensor media of one or more of the plurality of sensors includes one or more of a first agent selected from the group consisting of an antibody, an antigen receptor or an antigen covalently attached to at least a portion of the plurality of nanostructures and the sensor media of one or more others of the plurality of sensors includes one or more of a second agent selected from the group consisting of an antibody, an antigen receptor or an antigen covalently attached to at least a portion of the plurality of nanostructures, wherein the first agent is different front the second agent. In a number of embodiments, the sensor includes more than an agent selected from the group consisting of an antibody, an antigen receptor or an antigen covalently attached to at least a portion of the plurality of nanostructures.

In another aspect, a method of detecting an analyte which is an antigen or an antibody in a fluid sample includes exposing a sensor including a substrate and a sensor medium on the substrate to the fluid sample for a period of time (such that the fluid sample contacts the sensor medium). The sensor medium includes a plurality of nanostructures and One or more of at least one agent selected from the group consisting of an antibody, an antigen receptor or an antigen immobilized upon at least a portion of the plurality of nanostructures. The at least one agent is an antibody or an antigen receptor if the analyte is an antigen and the at least one agent is an antigen if the analyte is an antibody. The method further includes exposing the sensor to the fluid sample for a period of time, subsequent to exposing the sensor to the fluid sample, washing the sensor one or more times with a liquid of known ionic strength, and after washing the sensor, measuring an output of the sensor with the liquid of known ionic strength over or covering the sensor medium/sensor. The liquid having a known ionic strength may be chosen to have an ionic strength that is less than the fluid sample to increase sensitivity compared to output measured in the presence of the fluid sample. In a number of embodiments, the sensor is washed a plurality of times. Such washing may, for example, remove unbound species. In a number of embodiments, the liquid is a purified water. In a number of embodiments, the liquid electrolyte has resistivity greater than or equal to18.2 MΩ·cm.

In a number of embodiments, the plurality of nanostructures has an enriched semiconducting content. The plurality of nanostructures may, for example, has an enriched semiconducting content of at least 66%, 90%, 99% or 99.9%. In a number of embodiments., the measured variable is an electrical property change (for example, resistance or conductance). In a number of embodiments, the at least one agent is maintained or is immersed in a liquid phase. The sensor device may be thriller characterized as described herein.

As described above, in a number of embodiments, the plurality of nanostructures include carbon nanostructures. The plurality of carbon nanostructures may, for example, include or be composed of single-walled carbon nanotubes.

In a number of embodiments, the at least one agent is an antibody and the analyte is an antigen. The antigen may, for example, be an antigen of a pathogen selected from the group of a viral pathogen and a bacterial pathogen. The pathogen may, for example, be SARS-CoV-2, HIV, tuberculosis, syphilis, hepatitis (for example, hepatitis b or c), E. coli, Salmonella, Pseudomonas aeruginosa, Influenza, Staphylococcus aureus, or Streptococcus pyogenes, cytomegaloyirus (CMV), or Epstein-Barr virus (EBV), in a number of embodiments, the pathogen is SARS-CoV-2. The antigen may, for example, be a spike antigen (SAg) or a nucleocapsid protein antigen (NA) of SARS-CoV-2 and the antibody is anti-SAM-Cat-2 spike protein antibody (SAb) or anti-SARS-CoV-2 nucleocapsid protein antibody. In a number of embodiments, the antigen is a spike antigen (SAg) of SARS-CoV-2 and the antibody in the anti-SARS-CoV-2 spike protein antibody.

As described above, many other antigens, including non-pathogenic antigens may be detected by the sensors hereof. Such antigens include, for example, substance originating within the body (for example, hormones such as cortisol) and substances originating outside of the body (for example, drugs/opioids such as fentanyl/norfentanyl).

The sensor may, for example, be incorporated within a field effect transistor circuit or a chemiresistor circuit of electronic circuitry of the sensor. In a number of embodiments, the sensor is incorporated within a field effect transistor circuit and the liquid functions as a liquid gate.

The sensor device may, for example, include a plurality of the sensors. In a number of embodiments, the sensor media of one or more of the plurality of sensors includes one or more of a first agent selected from the group consisting of an antibody, an antigen receptor or an antigen covalently attached to at least a portion of the plurality of nanostructures and the sensor media of one or more others of the plurality of sensors includes one or more of a second agent selected from the group consisting of an antibody, an antigen receptor or an antigen covalently attached to at least a portion of the plurality of nanostructures, wherein the first agent is different from the second agent. In a number of embodiments, the sensor includes more than an agent selected from the group consisting of an antibody, an antigen receptor or an antigen covalently attached to at least a portion of the plurality of nanostructures.

The present devices, systems, methods and compositions, along with the attributes and attendant advantages thereof, will best be appreciated and understood in view of the following detailed description taken conjunction with the accompanying drawings.

### BRIEF DESCRIPTION OF THE DRAWINGS

FIG. 1a illustrates detection of SARS-CoV-2 Ag using SWCNT-based biosensors wherein a schematic structure of SA RS-CoV-2 is provided to demonstrate targeting proteins.

FIG. 1b illustrated schematically a liquid-gated SWCNT FET for detection of SARS-CoV-2 SAg and NAg. Interdigitated gold electrodes are configured as the source (So) and drain (Dr). Source-drain bias (V_{SD}) is 50 mV. Gate voltage (V_{g}) is applied through a silver'silver chloride reference electrode. Insets show SAb- and NAb-functionalized SWCNTs for specific detection of SAg and NAg, respectively.

FIG. 1e illustrates schematically pan embodiment of an antigen sensor hereof which is operable as a field effect transistor or FET.

FIG. 1d illustrates a perspective schematic view of an antigen sensor hereof which is operable as a chemiresistor.

FIG. 2 illustrates characterizations of SARS-CoV-2 antibody-functionalized SWCNT FET devices. (a) Microscopic image of SWCNT ET devices. (b) SEM image of SWCNT networks deposited on a FET device and (c) after SAb functionalization. The inset shows a zoom-in view of SAb-functionalized SWCNTs. (d) AFM image of SAb-functionalized SWCNTs. (e) Height profiles of SAb immobilized on the SWCNT and bare SWCNTs. (f) High-resolution XPS spectra of N 1 s of the bare SWCNT and antibody-functionalized SWCNT, and (g) C 1 s of the antibody-functionalized SWCNT with deconvolutions of the overall signal. (h) RBM region and (i) D and G peak regions of Raman spectra of the SWCNT during antibody functionalization. The RBM region was recorded using a 785 nm excitation laser. All spectra were normalized to the Si peak at 507 cm⁻¹. D and G peak regions were recorded using a 638 nm excitation laser. All spectra were normalized to the G peak at 1587 cm⁻¹. (j) LET transfer characteristics of an SWCNT FET device during NAb functionalization and (k) SAb functionalization,

FIG. 3 Illustrates detection of recombinant SARS-CoV-2 antigen proteins. (a) FET characteristic curves of SAb-functionalized SWCNT (SAb-SWCNT) FET devices upon exposure to increasing concentration of recombinant SARS-CoV-2 SAg. The inset shows the schematic illustration of PET configuration for SAg detection. (b) Calibration plot for SAg detection and nonspecific protein detection. (c) FET characteristic, curves of NAb-functionalized SWCNT (NAb-SWCNT) PET devices upon exposure to increasing concentration of recombinant SARS-CoV-2 NAg. The inset shows the schematic illustration of FET configuration for NAg detection. (d) Calibration plot for NAg detection and nonspecific protein detection. All data points plotted in the calibration plots are mean±SD. The number of devices (n) used for calculation is indicated in the parenthesis in the legend.

FIG. 4a illustrates clinical sample tests with SAb-functionalized FET devices. All data plotted are mean±SD,

FIG. 4b illustrates clinical sample tests with NAb-functionalized PET devices. All data plotted are mean±SD

FIG. 5. Antibody-functionalized sc-SWCNT-based LET biosensor configurations. a) schematic illustration for antibody functionalized sc-SWCNTs device; b) schematic illustration for antibody functionalized AuNPs-decorated sc-SWCNTs device.

FIG. 6. Surface morphology of cortisol antibody-functionalized FET sensors. SEM images for a) ab-sc-SWCNTs and b) ab-Au-sc-SWCNTs. AFM images of c) ab-sc-SWCNTs and d) Ab-Au-sc-SWCNTs.

FIG. 7. Raman spectroscopy characterization of ah-sc-SWCNT devices and ab-Au-sc-SWCNT devices. a) RBM region and b) G peak region of sc-SWCNTs before and after the immobilization of cortisol antibody using the direct attachment approach. c) RBM region and d) G peak region of sc-SWCNT, Au-sc-SWCNT and ab-Au-sc-SWCNTs. All G peaks were normalized to the G peak.

FIG. 8, Cortisol sensing in PBS. a) FET transfer characteristics of ab-sc-SWCNT devices upon adding increasing concentrations of cortisol. b) Calibration plot for cortisol sensing using ab-sc-SWCNT devices. The gate voltage chosen for calibration was −0.2 V. c) FET transfer characteristics of ab-Au-sc-SWCNT devices upon adding increasing concentrations of cortisol. d) Calibration plot for cortisol sensing using ab-Au-sc-SWCN I devices. The gate voltage chosen for calibration was −0.5 V. All data points plotted in the calibration plots are mean±SD. For both types of sensors, the number of devices tested were 6.

FIG. 9, FET characteristic curves with different cortisol concentrations for a) ab-sc-SWCNTs; b) ab-Au-sc-SWCNTs and linear calibration plot with different cortisol concentrations for e) ab-sc- SWCNTs and d) ab-Au-sc-SWCNTs.

FIG. 10. AFM images of a) bare sc-SWCNTs, b) norfentanyl antibody-functionalized sc-SWCNT device (norfentanyl ab-sc-SWCNT) via EDC/NHS (1-Ethyl-3-[3-dimethylaminopropyl]carbodiimide hydrochloride/N-hydroxysuccinimide) coupling approach; c) gold nanoparticle (AuNP)-decorated sc-SWCNTs; d) norfentanyl antibody-functionalized AuNP-decorated sc-SWCNTs (norfentanyl ab-Au-sc-SWCNT).

FIG. 11, Raman spectroscopy characterization of norfentanyl ab-sc-SWCNT devices and norfentanyl ab-Au-sc-SWCNT devices, a) G peak region and b) RBM region of sc-SWCNTs before (black) and after (red) the immobilization of norfentanyl antibody using the EDC/NHS coupling approach. c) Raman spectra of bare sc-SWCNTs (black), AuNP-decorated sc-SWCNTs (red), and norfentanyl antibody immobilized Au-sc-SWCNTs (blue).

FIG. 12. Norfentanyl sensing in PBS using fentanyl antibody (400 μg lint,) functionalized sc-SWCNT device via EDC/NHS coupling. a) AFM image of fentanyl antibody functionalized sc-SWCNT device with antibody concentration of 400 μg/mL. b) FET transfer characteristics of the fentanyl antibody-functionalized sc-SWCNT device upon exposing to different concentrations of norfentanyl in PBS. c) Calibration plot for norfentanyl sensing. (Results are Mean±SE, n=3 devices.)

FIG. 13. Norfentanyl sensing using fentanyl antibody-functionalized sc-SWCNT FET sensors. a) Calibration plot for norfentanyl sensing in PBS (square) and synthetic urine (circle) using fentanyl ab-sc-SWC NT devices. b) Calibration plot for norfentanyl sensing in PBS (square) and synthetic urine (circle) using fentanyl ab-Au-sc-SWCNT devices.

FIG. 14. Norfentanyl sensing in PBS. a) IT transfer characteristics of norfentanyl ab-sc-SWCNT devices upon adding increasing concentrations of norfentanyl. b) Calibration plot for norfentanyl sensing using norfentanyl ab-sc-SWCNT devices. c) FET transfer characteristics of norfentanyl ab-Au-sc-SWCNT devices upon adding increasing concentrations of norfentanyl. d) Calibration plot for norfentanyl sensing using norfentanyl ab-Au-sc-SWCNT devices. All data points plotted in the calibration plots are mean±SD. For both types of sensors, the number of devices tested were 8.

FIG. 15. Norfentanyl sensing in synthetic urine. a) FET transfer characteristics of norfentanyl ab-sc-SWCNT devices upon adding increasing concentrations of norfentanyl in 1000-fold dilution of synthetic urine. b) Calibration plot for norfentanyl sensing using norfentanyl ab-sc-SWCN1 devices in different dilutions of synthetic urine. c) FET transfer characteristics of norfentanyl ab-Au-sc-SWCNT devices upon adding increasing concentrations of norfentanyl in synthetic urine without dilution. d) Calibration plot for norfentanyl sensing using norfentanyl ah-Au-sc-SWCNT devices in different dilutions of synthetic urine. All data points plotted in the calibration plots are mean±SD. The number of devices tested was indicated in the parenthesis in panel b and d,

FIG. 16a illustrates a perspective view of an embodiment of a handheld sensor system hereof.

FIG. 16b illustrates a perspective view of an embodiment of the handheld sensor system of FIG. 16a hereof wherein a sensor module is removed from the system.

FIG. 16e illustrates a perspective, cutaway view of the handheld sensor system of FIG. 16a,

FIG. 16d illustrates schematically an embodiment of a voltage divider configuration of resistors for use in the electronic circuitry of the system of FIG. 16a, where a change in resistance is converted to a. change in voltage.

FIG. 16e illustrates schematically an embodiment of a Wheatstone bridge for use in the electronic circuitry of the system of FIG. 16a,

FIG. 161 illustrates schematically an embodiment of electronic circuitry for use in connection with the system of FIG. 16a.

### DETAILED DESCRIPTION

It will be readily understood that the components of the embodiments, as generally described and illustrated in the figures herein, ma be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. in the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in, detail to avoid obfuscation.

As used herein and in the appended claims, the singular forms “a,” “an”, and “the” include plural references unless the context clearly dictates otherwise. Thus, for example, reference to “a nanostructure” includes a plurality of such nanostructures and equivalents thereof known to those skilled in the art, and so Barth, and reference to “the nanostructure” is a reference to one or more such nanostructures and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise dearly contraindicated by the text.

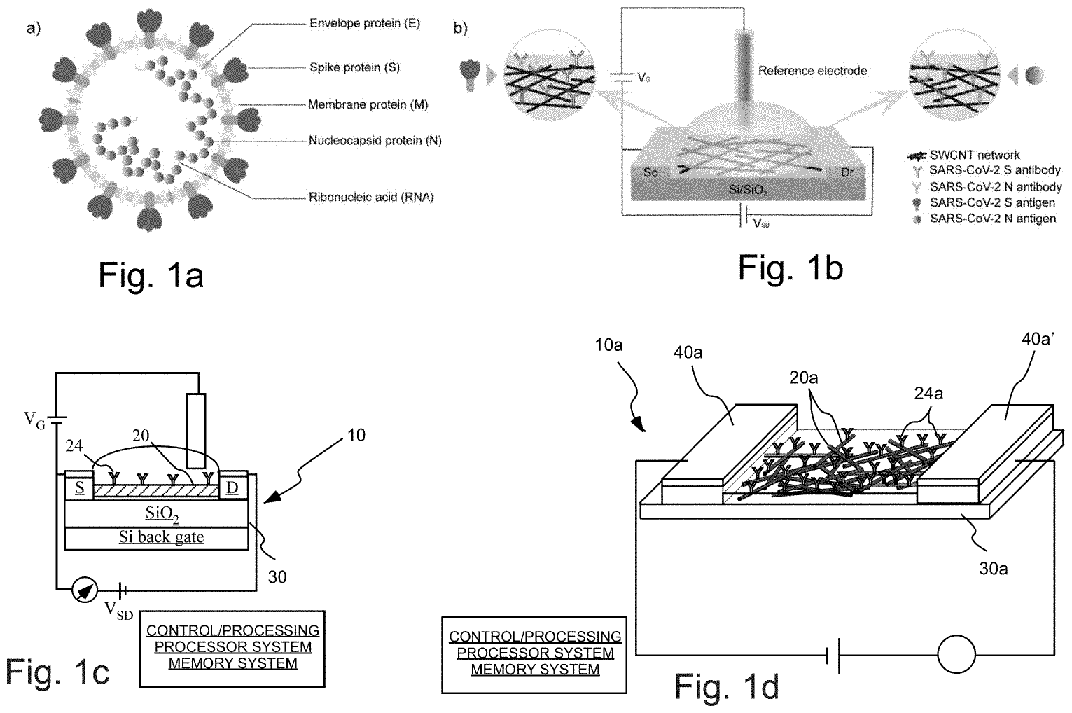

In a number of representative embodiments, the devises, systems, and methods hereof include nanostructure-based sensors or sensor systems which may, for example, be operated as a field-effect transistor (FET). The sensors are functionalized or decorated with specific binding chemistry to assess the presence of antigens (or antibodies), in a number of embodiments hereof a representative SARS-CoV-2 antigen (Ag) FET nanobiosensor includes a sensor including nanostructures such as high-purity semiconducting (sc) single-walled carbon nanotube (SWCNT) functionalized with a specific antibody (or a specific antigen receptor) to access the presence of one or more SARS-CoV-2 structural proteins (for example, spike protein (S antigen, SAg) and/or nucleocapsid protein (N antigen, NAg), see, for example, FIG. 1b-1d). High-purity sc-SWCNTs offer high on-state conductance and high on/off ratio for FETs, providing higher analytical sensitivity toward the target analyte compared to other carbon nanomaterials such as unsorted SWCNT and graphene. Moreover, SWCNTs are significantly less expensive and more widely available than CVD graphene films, thus lowering the cost of SWCNT FET sensors.

Although representative embodiments, devises, systems, and methods hereof are discussed in connection with sc-SWCNTs including immobilized antibodies or antigen receptors specific to SARS-CoV-2 antigens thereon to detect such SARS-CoV-2 antigens, other antibodies or antigen receptors associated, with antigens of pathogens other than SARS-CoV-2 as Well as nonpathogenic antigens may be immobilized on sc-SWCNTs or other nanostructures for use to detect such antigens. Moreover, antigens may be immobilized. on nanostructures to selectively detect the presence of various antibodies. Further, sensor assemblies hereof may be incorporated into electronic circuitry other than FET circuitry in sensors hereof.

The terms “electronic circuitry”, “circuitry” or “circuit,” as used herein include, but are not limited to, hardware, firmware, software, or combinations of each to perform a function(s) or an action(s), For example, based on a desired feature or need, a circuit may include a software controlled microprocessor, discrete logic such as an application specific integrated circuit (ASIC), or other programmed logic device. A circuit may also be fully embodied as software. As used herein, “circuit” is considered synonymous with “logic.” The term “logic”, as used herein includes, but is not limited to, hardware, firmware, software, or combinations of each to perform a function(s) or an action(s), or to cause a function or action from another component. For example, based on a desired application or need, logic may include a software controlled microprocessor, discrete logic such as an application specific integrated circuit (ASIC), or other programmed logic device. Logic may also be fully embodied as software.

The term “processor,” as used herein includes, but is not limited to, one or more of virtually any number of processor systems or stand-alone processors, such as microprocessors, microcontrollers, central processing units (CPUs), and digital signal processors (DSPs), in any combination, The processor may be associated with various other circuits that support operation of the processor, such as random access memory (RAM), read-only memory (ROM), programmable read-only memory (PROM), erasable programmable read only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

The term “controller.” as used herein includes, but is not limited to, any circuit or device that coordinates and controls the operation of one or more input and/or output devices. A controller may, for example, include a device having one or more processors, microprocessors, or central processing units capable of being programmed to perform functions,

The term “software,” as used herein includes, but is not limited to, one or more computer readable or executable instructions that cause a computer or other electronic device to perform functions, actions, or behave in a desired manner. The instructions may be embodied in various forms such as routines, algorithms, modules, or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software is dependent on, for example, requirements of a desired application, the environment it runs on, or the desires of a designer/programmer or the like.

As used herein, an “antigen” (sometimes abbreviated Ag) is any substance that induces a body to make an immune response against: the antigen. Antigens may include toxins, chemicals, viruses, bacteria, and other substances having an origin outside the body. Antigens may include a molecule or molecular structure which, for example, may be present on the outside of a pathogen, Antigens may also have an origin within the body. Body tissues and cells (for example, cancer cells) may also include antigens that cause an immune response. Further, hormones may be antigens. Antigens can be bound by antigen-specific antibodies or by a B-cell antigen receptor and typically trigger an immune response within the body. Antigens may be proteins, peptides (which are chains of amino acids) or polysaccharides (which are chains of monosaccharides or simple sugars). Lipids and nucleic acids may become antigens when combined with proteins and polysaccharides. An “antibody” (sometimes abbreviated as Ab and sometimes referred to as an is immoglobulin or 1 g), is a relatively large, Y-shaped protein that is used by the immune system to identify and neutralize foreign objects (including pathogenic bacteria and viruses). As used herein, an “antigen receptor” is a protein which selectively binds/interacts with an antigen.

In general, nanostructures are structures of intermediate size between microscopic and molecular structures. Nanostructures may, for example, have at least one dimension in the range of 0.1 to hundreds of nanometers. Many nanostructures have at least one dimension in the range of 1 to 100 nm. Nanotubes are, for example, considered two-dimensional nanostructures and may have a diameter in the range of, for example, 0.1 nm to hundreds of nm and a length that may be significantly greater that the diameter.

Chemically sensitive solid-state resistors (chemiresistors) and field effect transistors (FETs) hereof may, for example, exhibit room temperature liquid phase sensitivity to antigens (or antibodies) in, for example, a clinical sample such as a saliva, nasopharyngeal swabs, serum. plasma, bronchoalveolar lavage (BAL), or endotracheal aspirate (ETA). In general, any biological fluid/fluid sample may be analyzed to determine if the fluid sample includes an analyte above the detection level of a device hereof. In certain embodiments, it is also possible to analyze breath (for example, droplets of fluid from breath). in a nano structure-based FET device, one, for example, measures electrical current through nanostructures such as sc-SWCNT under an applied gate voltage. In chemiresistor devices, a gate voltage is not applied. In both types of devices, an electrical property (for example, conductance or resistance) of nanostructures such as nanotubes changes upon exposure to an analyte, thereby providing a sensor signal. Depending on the semiconducting nature of the nanostructures, application of a gate voltage can provide amplification of the sensor signal. Nanotubes such as single-walled carbon nanotubes or SWCNTs and, particularly sc-SWCNTs provide an ideal candidate for incorporation into extremely small and low power devices hereof because they demonstrate extreme environmental sensitivity, high electrical conductivity, and inherent compatibility with existing microelectronic fabrication techniques.

A schematic representation of an embodiment of an FET sensor device 10 hereof is set forth in FIGS. 1b and 1c, while an embodiment of a chemiresistor sensor device 10a hereof is illustrated in FIG. 1d. As described above, the illustrated sensor devices 10, 10a include a sensing medium material including one or more representative nanostructures. Such nanostructures include, for example, sc-SWCNTs 20, 20a. In a number of embodiments, the nanostructures are a network of sc-SWCNTs). Single walled carbon nanotubes are classified based on their electrical properties. Nanotubes may, for example, be considered to be either semiconducting or metallic. Nanotube synthesis process typically yields mix of both metallic and semiconducting nanotubes Purification steps are required to enrich the samples to be either mostly metallic or mostly semiconducting. Either mixed or purified nanostructures may be used in the sensor systems hereof. However, purified semiconducting nanostructures may provide improved, lower levels of detection and a wider dynamic range in devices, systems, and methods hereof. As used herein, the term “semiconductor enriched” in reference to nanostructures such as, sc-SWCNTs indicates that a semiconducting content of at least 66%, In a number of embodiments, the semiconducting content is at least 90%, at least 95%, at least 99%, or at least 99.9%. In general, a greater semiconducting content will result in a better output signal.

In single-walled carbon nanotubes, all carbon atoms are located, on the surface where current flows, making a stable conduction channel that is extremely sensitive to a surrounding chemical environment. Nanotubes and other nanostructures, including single walled nanotubes (SWNTs) such, as SWCNT's, have the ability to change conductance in response to interaction with analytes. This characteristic is, for example, implemented in a number of embodiments of systems 10 and 10a.

Various nanostructures other than SWCNTs are suitable for use in the present invention. Such nanostructures include, but are not limited to, multi-walled carbon nanotubes, graphene nanosheets and their derivatives (for example, reduced graphene oxide and holey graphene), nanowires, nanofibers, nanorods, nanospheres, nanoribbons (for example, interconnected nanoribbons of holey reduced graphene oxide) or the like, or mixtures of such nanostructures. Moreover, in addition to carbon, those skilled in the art will appreciate that the nanostructures of the present invention can be formed of boron, boron nitride, and carbon boron nitride, silicon, germanium, gallium nitride, zinc oxide, indium phosphide, molybdenum disulfide, silver, and/or other suitable materials. The formation and/or function of reduced graphene oxide and holey graphene compositions are, for example, discussed in U.S. Pat. Nos, 8,920,764, 9,482,638, and 10,801,982, and U.S. Patent Application Publication No. 2021/0122638, the disclosures of which are incorporated herein by reference.

As illustrated in FIGS. 1c and 1d, the sensing medium or material, including semiconducting sc-SWCNTs or a network of sc-SWCNTs 20 (or other nanostructures), may, for example, be disposed upon a substrate 30 (for example, silicon dioxide or quartz) and contacted by two conductive ON (for example, metallic—such as Au and/or Ti) electrodes representing a source (S) (a conductive electrode or terminal) and a drain (D) (a conductive electrode or terminal). In the operation of an FET circuit such as illustrated in FIGS. 1b and 1c, changes in electrical conductivity may, for example, be measured for an applied gate voltage. One may, for example, measure current flow between source (S) and drain (D) as a fraction of a swept/varied gate voltage range. In the liquid phase, the sensing material may, for example, be covered in liquid.

As described above, a chemiresistor sensor device such as device 10a need not include an applied gate voltage. In chemiresistor 10a the sensing medium or material, including nanostructures 20a, bridges the gap between two conductive electrodes 40a and 40a′ (for example, gold electrodes), which may be referred to a source and a drain. The sensing medium or material may alternatively be immobilized upon a set of interdigitated electrodes. The resistance/conductance between electrodes 40a and 40a′ can he readily measured. The sensing medium or material has an inherent resistance/conductive electrodes that is changed by the presence of the analyte. In a chemiresistor, a source-drain bias voltage may, for example, be swept through a range of voltages, and drain current may be measured.

As described above, in a number of embodiments, the sensing media or materials hereof include one or more antibodies, antigen receptors, or antigens immobilized upon the nanostructures thereof—for example, via covalent attachment of the antibody(ies), antigen receptor(s), or antigent(s) to the nanostructure. For example, in FIG. 1b-1d, antibodies 24, 24a are covalently attached to nanostructures 20, 20a to provide detection of antigens. It was surprising that covalent attachment of antibodies, antigen receptors, or antigens to semiconducting nanostructures such as sc-SWCNTs could be achieved. By covalently the antibody, antigen receptor, or antigen to the nanostructure, more robust sensors are achieved compared to various noncovalent immobilizations. One may, for example, readily remove (for example, via simple washing) unbound molecular species. Covalent linkage allows the achievement of increased sensitivities.

Problems in reproducibility may result from uncontrollable deposition and functionalization of nanostructures such as sc-SWCNT on the devices hereof, which may result in variance between devices. Significant device-to-device variability may, for example, make the calibration of devices more difficult, resulting in a requirement to calibrate each device individually. To improve reliability and standardized detection, controllable deposition and alignment of nanostructure/SWCNT flakes on devices hereof may be important in certain embodiments. For example, uniform SWCNT networks can be formed in the channel between electrode by ink jet printing or dip coating (for example, for fabricating thin-film transistors or TFTs) with <10% variability.

By integrating the anti-SARS-CoV-2 spike protein antibody (SAb) and anti-nucleocapsid protein antibody (NAb) with high-purity se-SWCNTs in representative studied. embodiments of biosensors hereof, the SARS-CoV-2 Ag FET biosensors demonstrated ultrasensitivity (at an order of magnitude lower than other sensors to date) and high analytical specificity toward SARS-CoV-2 SAg and NAg in calibration samples. Qualitative comparison of the NEAT and SARS-CoV-2 Ag FET devices indicated successful discrimination between positive samples and negative samples, indicating their potential in COVID-19 diagnostics. SWCNT FET sensors hereof, with functionalization of the anti-SARS-CoV-2 spike protein antibody (SA) and anti-nucleocapsid protein antibody, detected the S antigen (SAg) and N antigen (NAg), reaching a limit of detection of 0.55 fg/mL for SAg and 0.016 fg/mL for NAg in calibration samples. SAb-functionalized FET sensors also exhibited good sensing performance in discriminating positive and negative clinical samples, indicating a proof of principle for use as a rapid. COVID-19 antigen diagnostic tool with high analytical sensitivity and specificity at low cost.

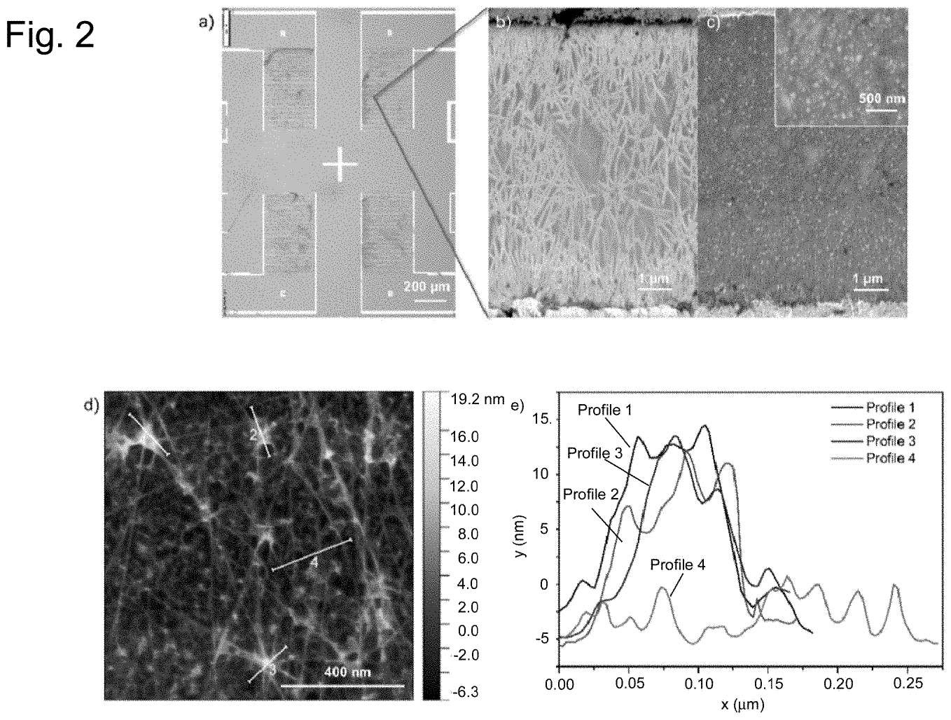

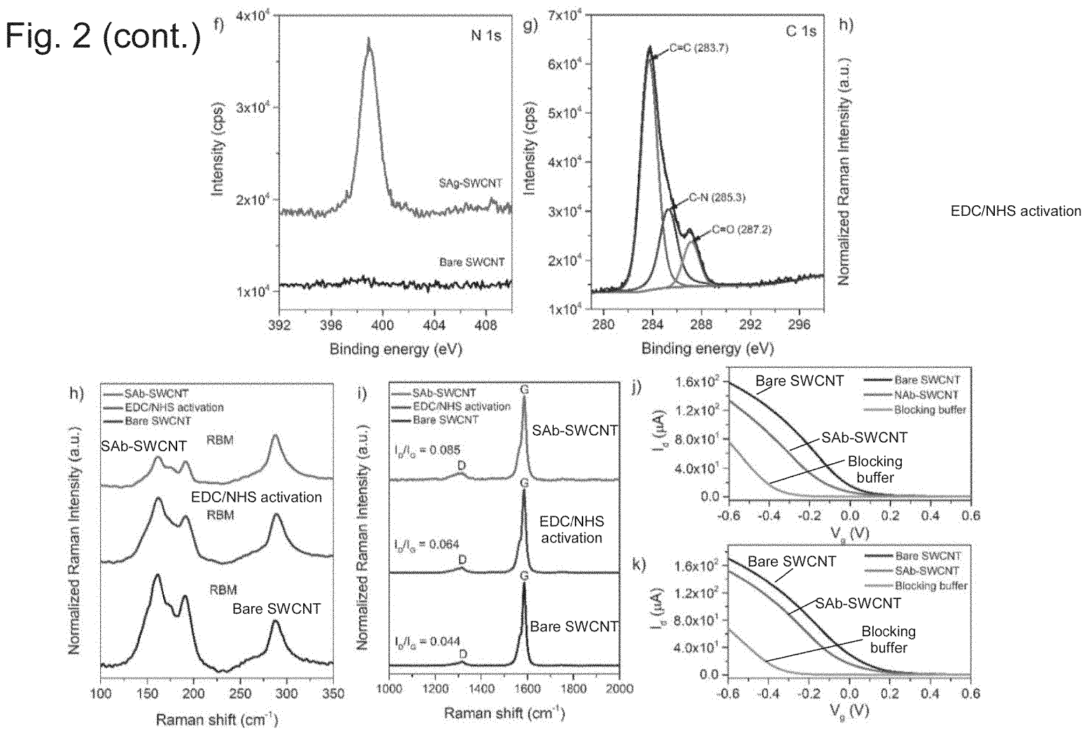

As shown in FIG. 2a, in a number of studied embodiments, the sensor chip included four FET devices with interdigitated gold source and drain electrodes patterned on a Si/SiO₂ substrate. The channel length was approximately 10 μm. To fabricate the SWCNT FET devices, sc-SWCNTs were deposited between interdigitated gold electrodes via dielectrophoresis/DEP forming dense and interconnected networks on the Si/SiO₂ surface (FIG. 2b). IR absorption spectroscopy was utilized to investigate the semiconducting content in the sc-SWCNTs, and the absence of the M₁₁ peak confirmed the high-purity semiconducting content.

The plurality of sensors/devices (four in the embodiment of FIG. 2a) may, for example, include identical sensor media. The sensor media of the sensors can also be different. In that regard, one or more of the sensors may include, a sensor medium that includes a different agent than the sensor media of the other sensors. More than one agent may, for example, be attached to the nanostructures of the sensor medium. In that regard, the attachment of more than one such agent to the sensor medium of a single sensor may facilitate detection of specific analytes/pathogens associated, for example, with a specific. disease (for example, pathogens associated with HIV). One sensor may, for example, include a sensor medium including antibodies, antigen receptors, and/or antigens immobilized on the nanostructures thereof. Such a sensor may, for example, be used in a screening process. A positive result in a screening process using such a sensor may, for example, prompt further testing.

SARS-CoV-2 SAb and NAb were conjugated onto sc-SWCNTs via EDC/sulfo-NHS ((1-Ethyl-3-[3-dimethyaminopropyl]carbodiimide hydrochloride/sulfo-N-hydroxysuccinimide) coupling between the carboxylic acid groups on the SWCNT sidewalk and the amine groups on the antibody. The EDC/sulfo-NHS coupling of antibodies on sc-SWCNTs was visualized using the enhanced green fluorescent protein (EGFP) antibody and EGFP. Green fluorescence was observable when EGFP was added to EGFP antibody functionalized device but not on bare SWCNTs without EGFP antibody, indicating the successful functionalization of EGFP antibody on the SWCNT surface. In EDC-NHS coupling chemistry, NHS and sulfo-NHS may, for example, be used to prepare amino-reactive esters of carboxylate groups for crosslinking and immobilization. Carboxylates (including a —COOH group) may be reacted to NHS or sulfo-NHS in the presence of a carbodiimide (for example, EDC to result in an NHS or sulfo-NHS ester, which may subsequently be reacted with primary amines (—NH₂) to form amide crosslinks.

The morphology of SAb on SWCNTs was characterized using scanning electron microscopy (SEM) and AFM (FIG. 2c,d). Me height profiles indicated a 12-15 nm increase in height after SAb immobilization (FIG. 2e). XPS provided complementary evidence for the integration of the antibody on SWCNTs. The high-resolution C 11s scan of the bare SWCNTs confirmed the presence of oxygenated defect sites on SW f. The appearance of the N 1s peak and C—N (285.3 eV) peak after antibody coupling also indicated successful conjugation of the antibody on SWCNTs (FIGS. 2f,g). Raman spectroscopy revealed the effect of antibody functionalization oar SWCNTs. Two major peaks were observed in the RIBM region. The peak that ranged from 125 to 225 cm⁻¹ decreased in intensity during functionalization, suggesting a preference of antibody functionalization on SWCNTs with larger diameters (FIG. 2h). Meanwhile, the I_{D}/I_{G} ratio increased from 0.044 to 0.085, indicating an increase in the degree of functionalization on SWCNTs due to the covalent bonding of antibodies to the SWCNTs (FIG. 2i), in FET transfer characteristics (FIG. 2j,k), the shift of threshold voltage toward more negative gate voltages and the decrease in the device conductance in the p-type region also revealed the successful functionalization of SAb and NAb on sc-SWCNTs. Further shift of the threshold voltage and decrease in the conductance were detected after the addition of blocking buffer (0.1% Tween 20 and 4% polyethylene glycol) to prevent nonspecific binding. Meanwhile, the gate leakage current was negligible compared with the ON state source-drain current, suggesting good insulation between the gate and source-drain electrodes; therefore, no encapsulation was required for Au electrodes.

The performance of SARS-CoV-2 Ag FET biosensors hereof was first investigated using BARS-CoV-2 Mg and NAg in calibration samples. All FET transfer characteristics were recorded via a liquid-gated FET configuration using nanopure water as the gating media to eliminate the impact of different ionic strengths on the sensing results. The present inventors have determined that it is desirable to maintain substantially the same ionic strength for all measurement (for example, FET measurements) to obtain an accurate calibration curve for the sensor. Different ionic strengths can lead to different sensor responses, which will lead to difficulty and/or inaccuracy in determining the sensor response arising from an antigen-antibody interaction, and eventually lead to inaccurate calibration for the sensor. In sensing experiments where all the measurements are performed in the analyte fluid sample or a sample including the fluid sample, as a result of different amounts of analyte present in the samples, differing amounts of analyte between samples may alter the ionic strength of the solution and consequently affect the sensor response. In a number of studied embodiments hereof, a purified water having a known and low ionic strength (for example, nanopure water) was used as the liquid for all measurements to maintain the ionic strength constant and eliminate the impact of different ionic strengths on the sensing results.

In that regard, in a number of embodiments, sensors hereof were inoculated with a sample of a biological fluid or a stimulant therefor (for example, phosphate buffer saline or PBS). After a period of time of inoculation (for example, approximately 2 minutes in a number of studies hereof), a measurement was taken using a liquid electrolyte (a liquid gating electrolyte in an FET sensor) of a known ionic strength which was lower than the fluid sample (ionic strength be expressed as conductivity or resistivity) over/covering the sensor medium. Nanopure water, for example, with resistivity >18.2 MΩ·cm was used in a number of embodiments. Improved results (including improved reproducibility, accuracy and/or a lower detection level) were achieved by washing sensors hereof with a low-ionic-strength fluid such as the liquid electrolyte to remove any unbound protein before measurement in the liquid electrolyte. In a number of embodiments, the devices were then washed three times with nanopure water before measurement in nanopure water as the liquid electrolyte.

Sensitivity can be increased in sensor hereof through use of a low-ionic strength fluid through reducing or eliminating the Debye screening effect. As known in the art, Debye screening length (λ_{D}) is a measure of a mobile charge carrier's electrostatic effect and how far it screens out electric fields in plasmas and other conductors. Debye screening length can be calculated using the following equation:

$${λ}_{D} = {( 4 π {L}_{B} {\sum }_{i} {n}_{i} {z}_{i}^{2} )}^{- 1 / 2}$$

In the above equation, L_{B} is the Bjerrum length (0.7 nm), and nᵢ and z₁ are the number concentration and the valence of ion species, i. Because of the limitation of Debye screening length, only the interactions within the Debye screening length can be probed. Therefore, for electronic biosensors, the Debye screening effect may be an issue because mobile ions present in biological samples screen charges from the target molecule, significantly reducing sensor sensitivity.

From the above equation, it can be concluded that λ_{D} can be tuned by altering the ionic strength (I=½Σᵢnᵢzⱼ²) of the solution. In that regard, by lowering the ionic strength of the liquid used for sensor measurements, the sensors can probe the interactions near the suffices more efficiently, and therefore provide better sensitivity.

For example, the length of a typical immunoglobulin 0 or IgG type antibody is around 10-15 nm. Therefore, to sense the binding between the antigen and the antibody, it is desirable to use a liquid that has a Debye screening length longer than 10 nm for the measurements. In that regard, it is desirable that the screening length exceed the length of the immobilized antibody, an antigen receptor or an antigen hereof. According to the above equation for the Debye screening length, the ionic strength of the liquid should be lower than 0.94 nmol/L to provide a Debye screening length longer than 10 nm. One may thus, readily determine a desirable ionic strength for liquids used in measurement methodologies of sensors hereof. As set forth above, nanopure water was used as the liquid for liquid-gated FET measurements. As a result of the minimal number of ions present in nanopure water (low ionic strength), it was estimated the nanopure water has a Debye screening length of around 100 nm, thus ensuring the detection of the binding between antigen and antibody that occurs near the sensor surfaces and providing improved sensitivity as compared to measurements in the presence of the fluid sample.

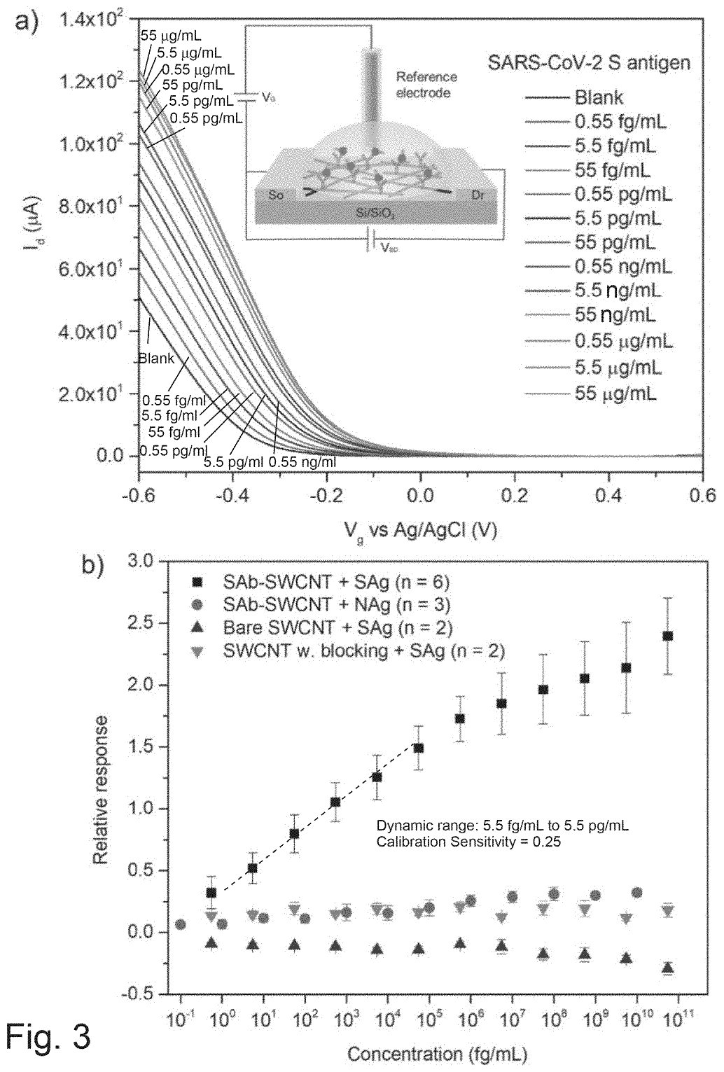

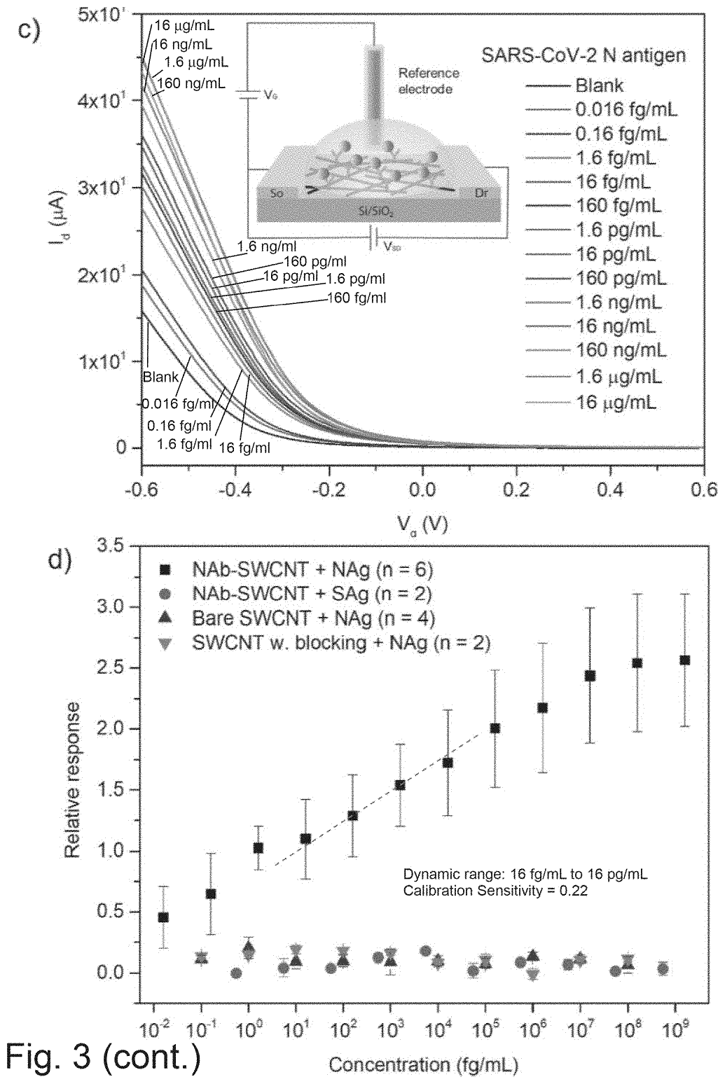

FIG. 3a shows the curve of SARS-CoV-2 SAb-functionalized FET devices for the detection of SAg. A shift toward more positive threshold voltages can be observed in the FET characteristics when only 0.55 fg/mL. SAg was introduced to the device. The threshold voltage shifted further toward more positive values with increasing concentration of SAg. Without limitation to any mechanism this positive shift of the I-V_{g} curve can be attributed to the introduction of negatively charged SAg, (pI=6.24) near the SWCNT surface, inducing additional hole carriers, thus p-doping the SWCNTs. The calibration curve (FIG. 3b) was constructed by plotting; the relative response (R, R=ΔI/I₀, where ΔI=I_{d}−I₀ and I₀ is the drain current in nanopure water before antigen exposure at −0.5 V_{g}) against concentrations of SAg in a logarithmic scale, where the dynamic range (see red broken line in FIG. 3b) of the SAg sensor can be determined to be 5.5 to 5.5 pg/mL and the calibration sensitivity, defined as the slope of the linear region of the calibration curve, to be 0.25 by fitting the calibration curve. Control experiments with NAg demonstrated the high specificity of the SAb-functionalized devices. The low relative response of the bare SWCNT FET sensor with or without blocking toward SAg further indicated that the responses of the SAb-functionalized device toward SAg were indeed induced by the specific antigen—antibody interaction.

Similar to SAg detection, PET transfer characteristics of NAb-functionalized FET devices showed a consistent shift of the threshold voltage toward the more positive region with increasing NAg concentration (FIG. 3c) due to the electrostatic gating effect. However, with the addition of positively charged NAg, and without limitation to any mechanism, the sensing mechanism is likely due to the neutralization of the positively charged antibody upon NAg binding. By fitting the calibration curve, the dynamic range is found to be 16 fg/mL to 16 pg/mL and the calibration sensitivity is 0.22, displaying similar sensing performance to SAg detection. Meanwhile the NAb-functionalized FET devices showed minimal responses to nonspecific proteins, and SWCNT FET devices without NAb conjugation also did not respond to the addition of NAg, exhibiting the high specificity of the NAg sensor (FIG. 3d).

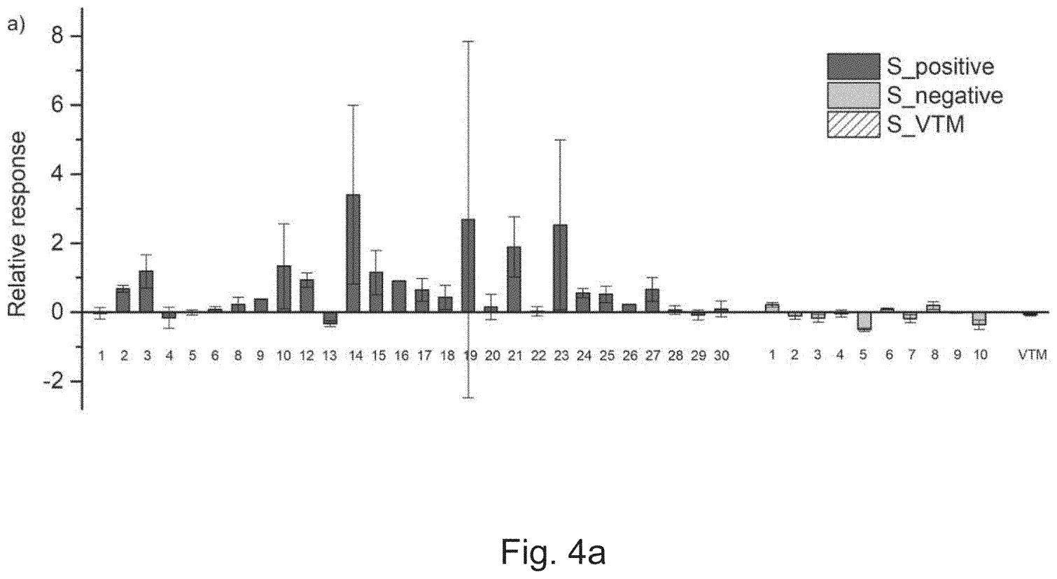

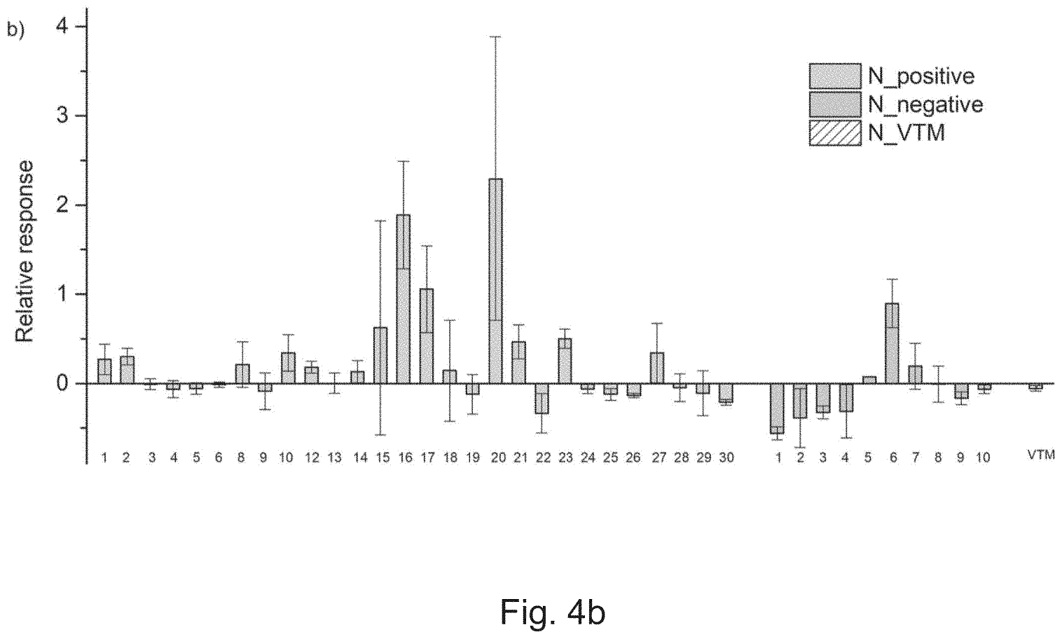

SARS-CoV 2Ag FET biosensors hereof were then tested with clinical samples. A total of 28 NAAT positive samples and 10 NAAT negative samples were tested using both SAb-and NAb-functionalized FET biosensors. All clinical samples were nasopharyngeal swabs suspended in the viral transport medium (VTM), and the viral load of each sample was measured by approved FDA ELLA NAAT assays. The existing literature indicates that the NAAT and antigen detection are biologically well correlated but not 100% concordant. The NAAT can detect RNA before a significant antigen is produced; additionally, the temporal course of the antigen versus RNA clearance after active infection is unclear. FIGS. 4a and 4b, respectively, summarize the relative responses of both SAb and NAb-functionalized sensors for all samples and blank VTM. A total of 23 out of 28 non-optimized SAb-functionalized devices responded positively to NAAT positive samples, consistent with what was observed previously, yielding a 17.8% false negative rate compared to the EUA NAAT. On the other hand, 7 out of 10 gave negligible or negative responses toward NAAT-negative samples. The blank VTM, which contains Hank's balanced salt solutions, fetal bovine serum, gentamicin, and amphotericin B, only induced small negative relative response of the devices. Therefore, the negative responses and false positive responses from the NAAT negative samples may come from other biological species collected from the nasopharyngeal swab or represent the continued presence of the antigen after RNA clearance. The results indicate that the SAb-functionalized FET biosensor has the potential to work as a rapid clinical SARS-CoV-2 antigen detection diagnostic.

The non-optimized NAb-functionalized FET devices demonstrated less effective discrimination between positive and negative samples as 15 out of 28 positive samples produced positive responses. Moreover, the induced responses are, in general, lower than those of SAb-functionalized devices. The less of detection of NAA may be attributable to the lower concentration of available NAg present in the clinical samples. While SAg is on the surface of SARS-CoV-2 virus, NAg, whose primary function is to form a capsid to protect the viral genome and enter the host cell, is released only when the virus enters the host cell. Without further sample processing, NAg might be limited within the virus or infected cell and therefore cannot be detected. For negative samples, although NAb-functionalized devices yielded the same false positive rate as SAb-functionalized devices, they had increased comparative responses to negative samples and blank VTM. This may indicate a higher susceptibility of NAb-functionalized devices to nonspecific binding of biomolecules or cross-reactivity of the antibody with other proteins. However, the results for NAg detection are antibody-specific, Better sensing performance might, for example, be achieved using a different Nab and/or otherwise optimizing the devices.

Thus, in a number of representative embodiments hereof SARS-CoV-2 antibody-functionalized SWCNT-based FET biosensors were used to assess the presence of the SARS-CoV-2 antigen in less than 5 min and at a few cents per test. Both SAb- and NAb-functionalized FET biosensors exhibited ultrasensitivity and high specificity toward their specific SARS-CoV-2 antigen in calibration samples. The limit of detection is determined to be 0.55 fg/mL for SAg and 0.016 fg/mL for NAg. Our SARS-CoV-2 Ag FET biosensor also achieved rapid detection of SARS-CoV-2 antigens in clinical samples without prior sample processing, and the results suggested that the studied SAb-functionalized devices performed better than NAb-functionalized devices in discriminating COVID-19 positive and negative samples collected from nasopharyngeal swabs and are less susceptible to nonspecific species present in the clinical samples. Furthermore, the sc-SWCNT FET detection assay approach hereof opens the opportunity for multiplex detection of not only viral antigens but also antibodies recognizing these antigens.

In general, further optimization may include determining an improved or optimal antibody for SARS-CoV-2 Ag detection. Sensor devices functionalized with, for example, S and N antibodies and their variants obtained from difference sources may be tested using both SAg and NAg calibration samples and COVID-19 NAAT-confirmed samples. The sensing performance (limit of detection, dynamic range, calibration sensitivity, etc.) of each type of sensor device may be assessed using the calibration curve constructed from testing with calibration samples. Qualitative comparison of results of NAAT-con firmed samples and sensing results may also be performed to determine the best-performing sensor.

To quickly screen the various biosensor and sensor array chemistries, an automated sensor test system may, for example, be used, The test system may, for example, include a pipette robot that performs the liquid handling, a source meter unit (SMU) that measures the output (for example, FET transfer curves), and system switch that enables multiplexing. A software (for example, Python) script may be used to synchronize the liquid handling and measuring the sensor output traces in addition to batched data processing of the sensor measurements.

Machine learning for the identification of serological signatures of SARS-CoV-2 infection may be used. To improve the reliability of sensing results, one may, for example, use a multiplex SARS-CoV-2 Ag sensor utilizing sensor array decorated with various specific SAS-CoV-2 Ag receptors to detect SARS-CoV-2 Ag. FET characteristics of the sensor array may be recorded in both calibration samples and NAAT-confirmed clinical samples, and differential array response may be used to generate statistically a fingerprint of SARS-CoV-2 Ag response.

Output (for example, FET trace) datasets may be used as the basis for training machine learning algorithms such as k-nearest neighbor, support vector classifier, deep learning, and random forest. Classification accuracy as measured by JO-fold cross-validation may, for example, be used as the validation metric. Cross-validation accuracy is desirably above 90%. Once the 90% accuracy is achieved through acquisition of a robust data set and optimization of the sensor array chemistry, blinded positive and negative samples may be tested to further validate the accuracy of the sensor array measurements and the model. Such optimization procedures and/or other optimization procedures may be carried out with sensors hereof for antigens other than SARS-CoV-2 Ag.

In that regard, there are many other antigens, including non-pathogenic molecules which may be detected by the sensors hereof. Such antigen molecules/substances may originate internally or externally of the body. Cortisol, for example, is a steroid hormone reflecting stress level in human body, high cortisol levels are usually associated with high stress level, which can cause physical problems such as hypertension, while low cortisol levels can be linked to fatigue, low blood pressure, and low blood sugar. Cortisol levels can be monitored non-invasively in body fluids such as urine, saliva and sweat. Detection of cortisol in sweat can be further engineered into wearable devices, which conveniently provide real-time monitoring of cortisol level. However, selectively monitoring cortisol in human sweat represents analytical challenge due to its low concentrations compared to other components such as sodium chloride. The design of portable or wearable biosensors to monitor cortisol levels in sweat (liquid environment) necessitates the selection of sensor materials that are sensitive to cortisol concentration change with low cross-sensitivity to other sweat components which are present at higher concentrations.

Opioids include compounds that are extracted from the poppy seed, as well as synthetic. compounds with similar properties that can interact with opioid receptors in the brain. Among them, fentanyl is a potent synthetic opioid that is used as a pain reliever and as an anesthetic. It is approximately 50-100 times more potent than morphine. However, because of their pharmacological effects, the overdose of fentanyl can cause difficulties in breathing, and can lead to death, and the death rates due to opioids overdose in the US increased substantially between 1999-2019. In human body, fentanyl gets metabolized to norfentanyl via oxidative N-dealkylation and to 4-ANPP via hydrolysis. Norfentanyl, as the primary inactive metabolite of fentanyl, can be detected in body fluids with wider detection window. The development of biosensors using functionalized carbon nanotubes for ultrasensitive norfentanyl sensing in body fluid such as urine, serum and sweat can help combat the opioid overdose epidemic.

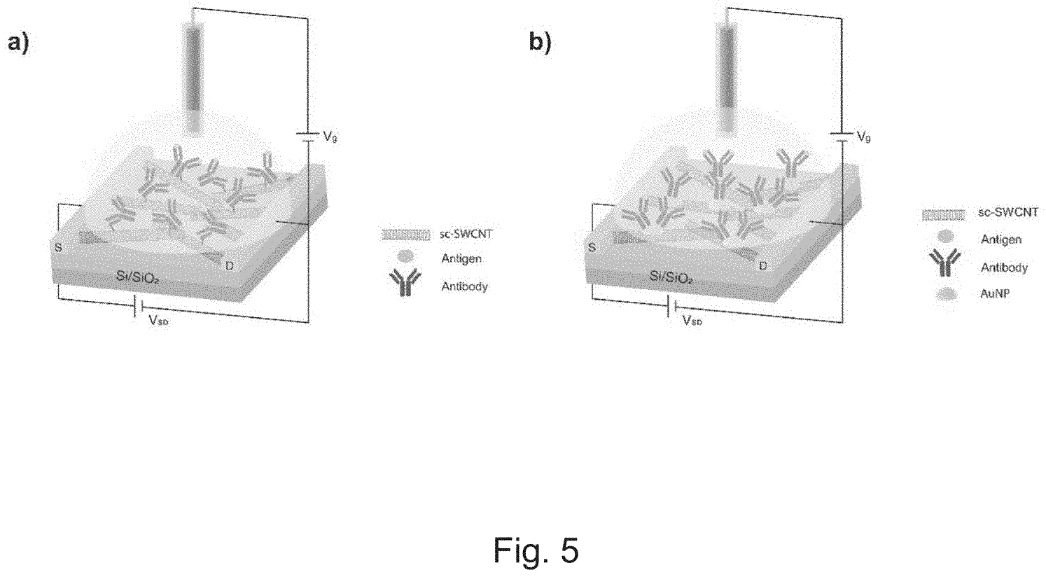

The efficacy of sensors hereof was also demonstrated in studies of nonpathogenic antigens such as cortisol and norfentanyl. FIG. 5 demonstrates the two FET sensor configurations designed and fabricated. Although both having sc-SWCNTs as the transducing layer, one had antibody directly attached to sc-SWCNTs via EDC/NES coupling by forming amide bonds between the carboxylic acid groups present on sc-SWCNTs and the amine groups on the antibodies (FIG. 5a), and the other one had a layer of AuNPs nucleated onto sc-SWCNTs, and the antibody was attached to the AuNPs surface via thiol-Au interactions (FIG. 5b).

A chemical bond can be formed between sulfur and gold. Agents hereof may be bonded (for example, covalently attached) to the gold to attach the agents to the nanostructures hereof. For example, gold nanoparticles can anchor on the defect sites on SWCNTs. On the other hand, a gold nanoparticle-decorated carbon nanotube is an important hybrid material based on carbon nanotubes that provide unique properties for biosensing. The gold nanoparticles can be viewed as a linker between the antibody and the nanostructures/SWCNTs hereof.

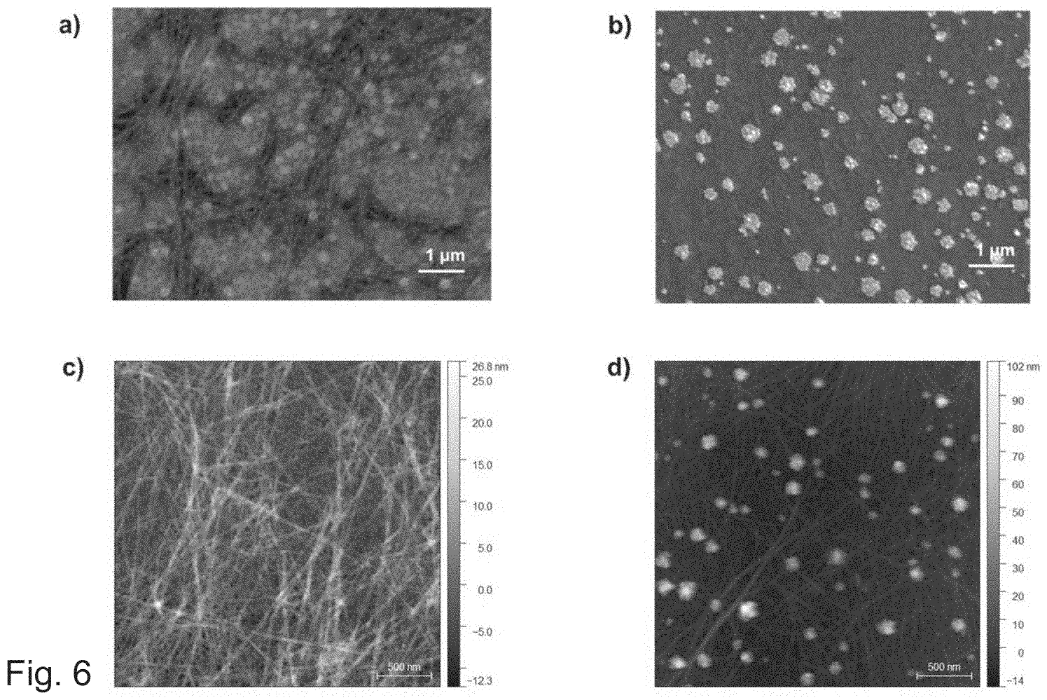

The surface morphology of the devices was characterized using SEM. Sc-SWCNTs were deposited between the interdigitated gold electrodes and formed a dense network. In embodiments with AuNP decoration, discrete AuNPs anchored to sc-SWCNTs with sizes range from 30-100 nm. After the immobilization of cortisol antibody, small nucleation of cortisol antibody on sc-SWCNTs surface was observed in embodiments using the EDC/NHS procedure (FIG. 6a). FIG. 6b shows the patterns of antibody decorated AuNPs that anchored at the defects of sc-SWCNT& Without limitation to any mechanism, the rough edges of the AuNPs might indicate the presence of antibody, similarly to other examples of protein binding. AFM was then used to provide height information of the attachment of antibodies on the sc-SWCNTs (FIG. 6c and d). By using the direct coupling (E DC/NHS) approach, cortisol antibodies were immobilized along the carbo nanotubes, resulting in a 10-13 nm increase in the height profile. The binding of antibodies on AuNPs resulted in an increase in the height of AuNPs from 46+12 nm to 56+16 nm.

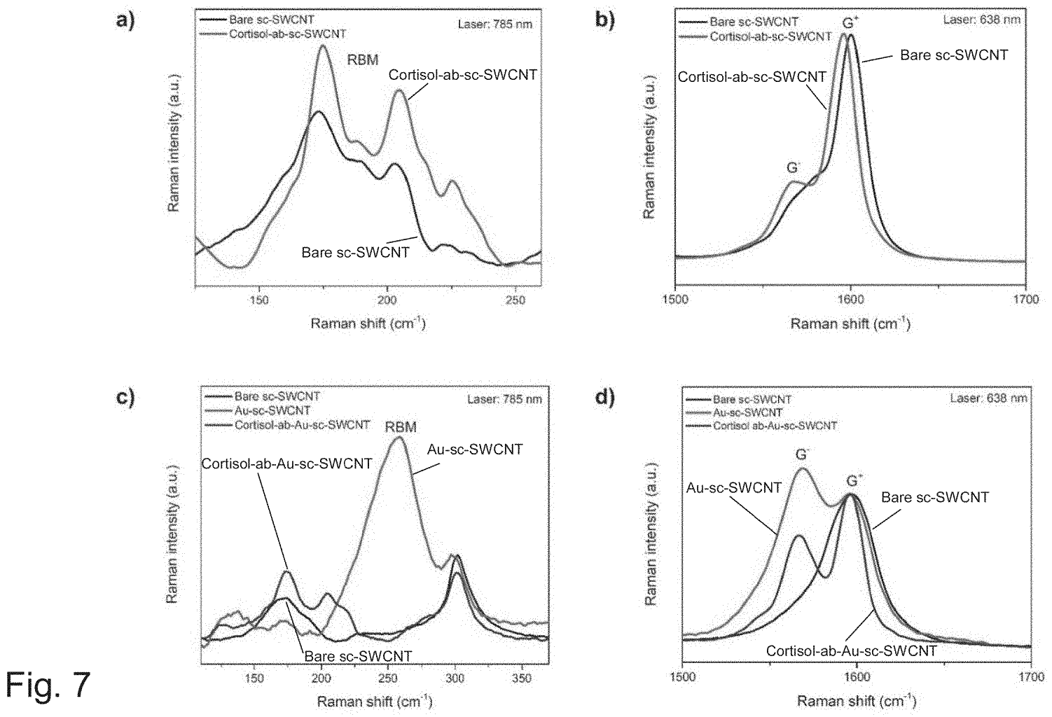

Raman spectroscopy provided further evidence for characteristics of ab-sc-SWCNT devices and Au-ab-sc-SWCNT devices. FIG. 7a and e showed that antibody attachment to the surface of both types of sensors caused the radial breathing mode (RBM) peak of sc-SWCNTs shift toward higher frequency, which may be attributed to the change in resonance excitation energy due to functionalization. The direct attachment of the cortisol antibody to the sc-SWCNTs led to a redshill of the Gr⁺ peak, indicating an electron transfer process happened toward the sc-SWCNT network. Moreover, a further splitting of the G peak was observed in both ab-sc-SWCNT devices and ab-Au-sc-SWCNT devices as sc-SWCNTs were further functionalized (FIG. 7b and d), This could be explained by the change of strain perpendicular to the tube axis res u I tin g from functionalization on the sc- SWCNTs.

The sensing performance and sensing mechanism of both ab-sc-SWCNTs and ab-Au-sc-SWCNTs devices were first investigated using cortisol solutions in phosphate buffered saline (PBS). The concentration of cortisol solutions ranges from 1.0×10⁻¹³M to 1.0×10⁻⁷M, as cortisol aqueous solution saturates at 4.4×10⁻⁷ M at room temperature. For all liquid-gated FET measurements, nanopure water was used as the gating liquid as described above, thereby circumventing the limitation of Debye screening effect.

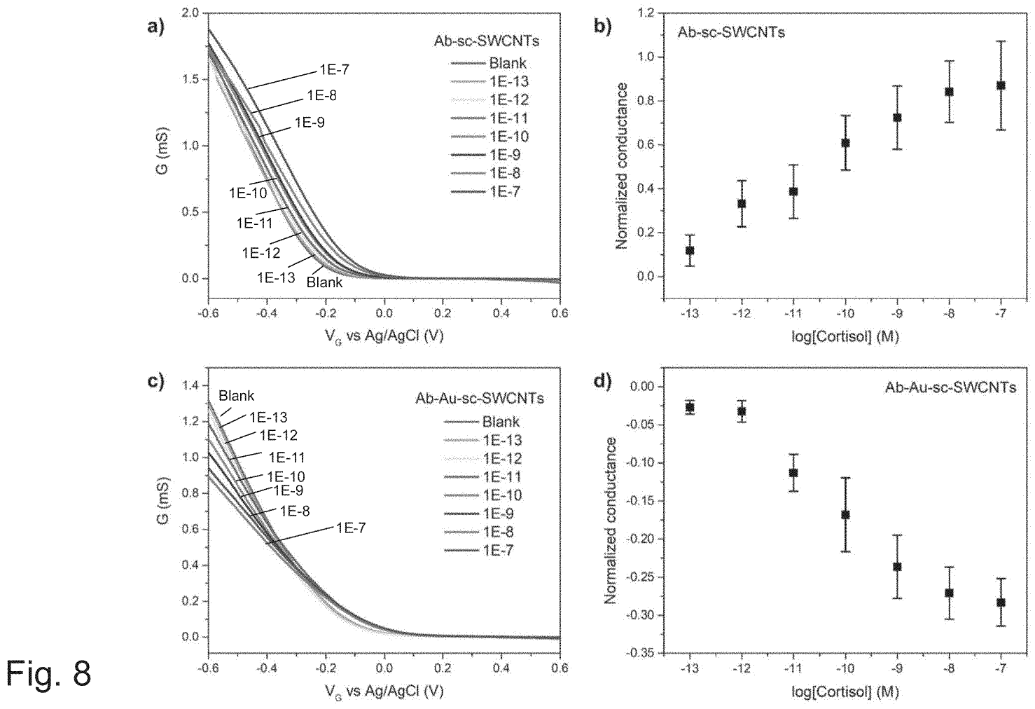

The raw FET characteristic curves for both types of sensors presented in FIG. 8a and FIG. 8c exhibited typical transfer curves for p-typo. FETs. However, by plotting the calibration curve using the normalized conductance at a given voltage, it is noticeable that the two types of sensors responded to cortisol in opposite trend (FIGS. 8b and 8d). For ab-sc-SWCNT devices, the FET characteristic curves shifted toward more positive voltages with increasing concentration of the analyte, resulting in an increasing trend of the normalized conductance. The increasing trend of the sensor response may be attributed to the electrostatic gating mechanism. While cortisol is charge neutral under physiological conditions, it binds to its antibody through forming hydrogen bonds with the tryptophan residues, located in the binding pocket, with tryptophan being the hydrogen bond donor. As a result, the formation of the hydrogen bond could lead to a charge redistribution, making the binding sites of the antibody more negatively charged, and the whole antibody less positively charged, therefore p-doping the sc-SWCNTs and shifting the FET characteristic curves toward more positive gate voltages,

As illustrated in FIG. 8d, an opposite trend resulted in the ab-Au-sc-SWCNTs device. With the concentration of cortisol increasing, the device showed a decreasing trend of the conductance of the sensor. Without limitation to any mechanism, this might be explained with. Schottky barrier change, which is another type of sensing mechanism. Schottky barrier occurs at the junction of metal and semiconductor, and in the ab-Au-sc-SWCNTs sensor, Schottky barrier forms at the junction of AuNPs and sc-SWCNTs surface. The work function of AuNPs could be altered as cortisol interacted with cortisol antibody, which results in Schottky barrier change, and thus reflected a different pattern of FET characteristic change.

The specificity of those two types of sensor devices were evaluated via comparison devices without cortisol antibody decoration for the sensing of cortisol. Compared with the devices decorated with antibodies, the conductance change was almost negligible on sc-SWCNTs and Au-sc-SWCNTs devices at different concentrations. Without limitation to any mechanism, it may be concluded that cortisol molecules would not interact with the CNT hybrids without antibody decorations, and the significant changes in conductance on ab-Au-sc-SWCNTs/ab-sc-SWCNTs devices were only associated with the specific interaction between cortisol and antibody molecules.

The cross-relativity with corticosterone was also studied. The cortisol antibody used in the present studies has a >90% cross-relativity with corticosterone due to the structural similarity between cortisol and corticosterone. Studies have shown that the 20-keto and 21-hydroxyl groups are essential for the binding between cortisol and the tryptophan residue located on the antibody, and corticosterone shares the same structure in this area. The sensors with were tested with corticosterone in PBS. Similar sensing behaviors were observed for both types of sensors. The findings further confirmed the hypothesis that the sensing of cortisol arises from the formation of hydrogen bonds between cortisol and the antibody thereby changing the charge density near the antigen binding sites on the antibodies.

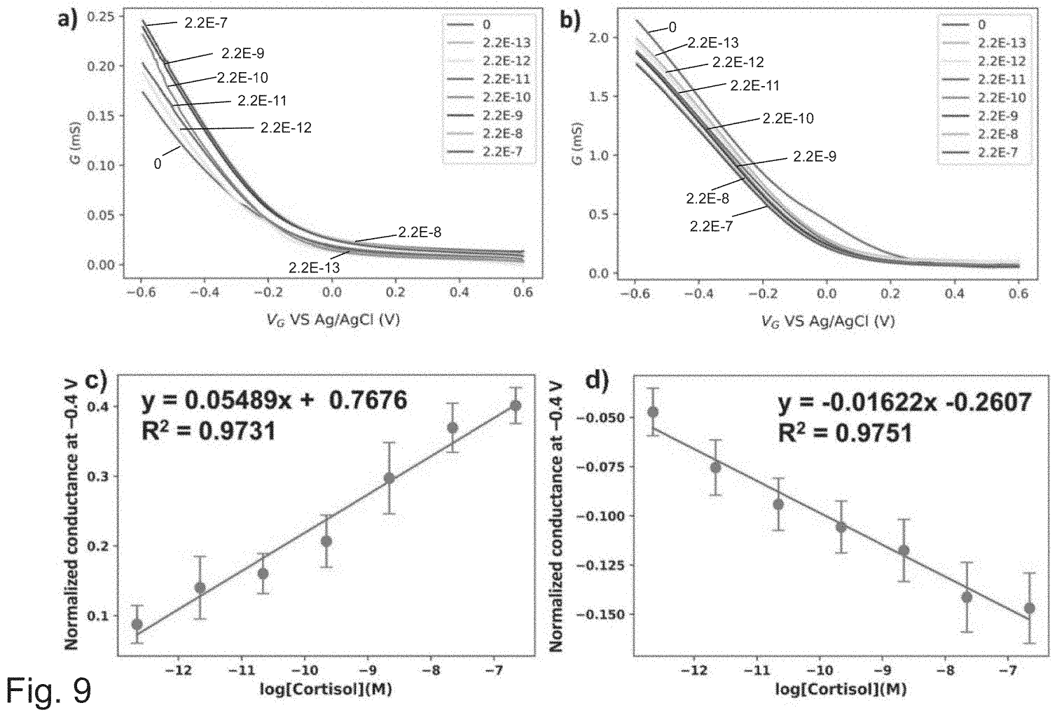

To study the sensor performance of both types of cortisol sensors in a complex environment, the sensors were tested in artificial sweat containing different concentrations of cortisol. As shown in FIG. 9, similar sensing behaviors were observed for both type of sensors. As also illustrated in FIG. 9, the conductance in ab-Au-sr-SWCNTs devices is much higher than in ab-sc-SWCNTs because of the AuNPs decoration. The charge transfer occurs from sc-SWCNTs to AuNPs, which caused the depletion of electron density of sc-SWCNTs. The normalized conductance at −0.4 V was selected as the sensor response to perform sensor calibration. The calibration plots for ab-sc-SWCNTs and ab-Au-sc-SWCNTs devices can be found in FIG. 9c and FIG. 9d. The error bars are the standard deviation from 4 different devices on the chip. The error bars are the standard deviation from 4 different devices on the chip. The coefficients of variation for concentrations front 2.2×10^{-13 to} 2.2×10⁻⁷, For ab-sc-SWCNTs devices, the coefficients, are 0.3090, 0.3205, 0.1790, 0.1809, 0.1720, 0.0955, 0.0643. For ab-Au-sc-SWCNTs devices, the coefficients are −0.2553, −0.1859, −0.1410, −0.1250, −0.1342, −0.1253, 0.1218. The slope of the calibration curve of ab-sc-SWCNT devices is determined to be 0.05, which is five times higher than that of ab-Au-sc-SWCNT devices, indicative of a significant higher sensitivity of the ab-sc-SWCNT devices. A reason for such a difference in the sensitivity may lie in the distance between the receptor (i.e., cortisol antibody) and the transducing layer (i.e., sc.-SWCNTs). For ab-sc-SWCNTs, the receptor and transducing layer are covalently linked, whereas for ab-Au-sc-SWCNTs, the distance is the height of AuNPs, which is around 46 nm. Consequently, the shorter distance between the receptor and the sc-SWCNTs led to the higher sensitivity of ab-sc-SWCNT sensors.

It was thus observed that directly attaching antibody to sc-SWCNTs provides the sensor with a much better sensitivity towards cortisol (five times more sensitive), and a slightly better limit of detection or LOD (10⁻¹⁴ M versus 10⁻¹³ M) than having an extra layer of AuNPs in between the antibody and the sc-SWCNT. A good LOD and linear responses provide the cortisol sensors significant potential to, for example, be incorporated with a microfluidic device as wearable device, and to provide real-time monitoring of cortisol level in human sweat.

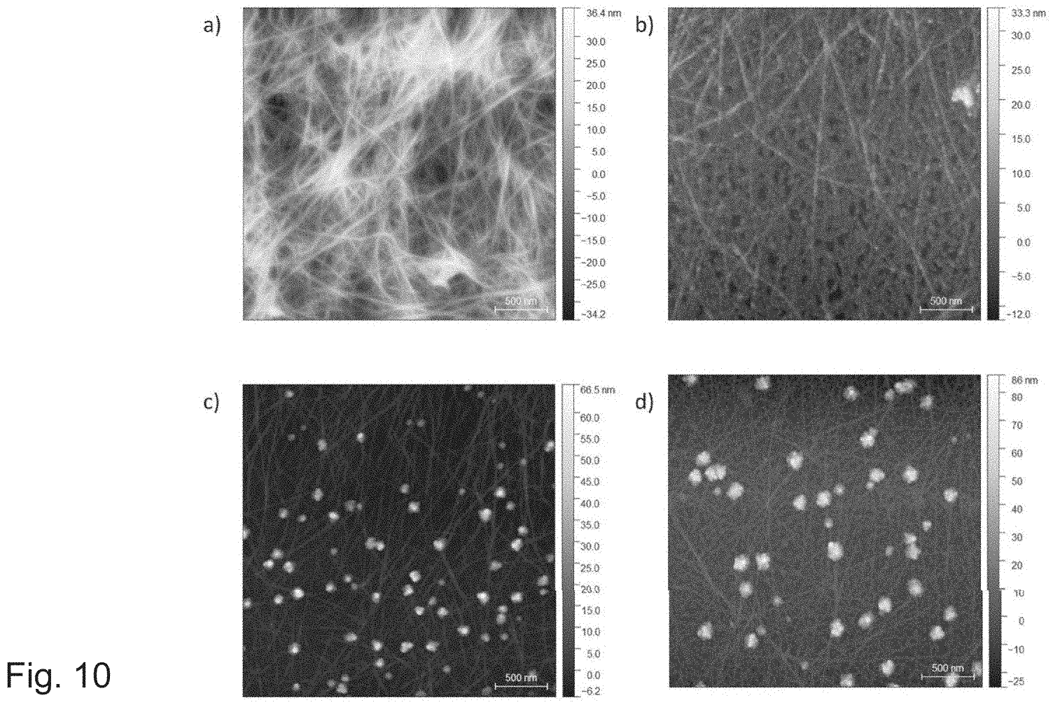

The same or similar approaches as those described above were also applied to investigate the norfentanyl sensing performance of norfentanyl antibody-functionalized sc-SWCNT-based FET biosensors. Norfentanyl antibody was immobilized on the sc-SWCNTs via the two different approaches discussed above to detection of cortisol (that is, antibody directly attached to sc-SWCNTs via EDC/NHS coupling, and antibody attached to AuNPs nucleated onto sc-SWCNTs, wherein the antibody was attached to the AuNPs surface via thiol-Au interactions). The surface morphology of the FET devices was characterized using AFM. As shown in FIG. 10, for both types of devices, a˜10 nm increase in height can be observed after the attachment of antibody.

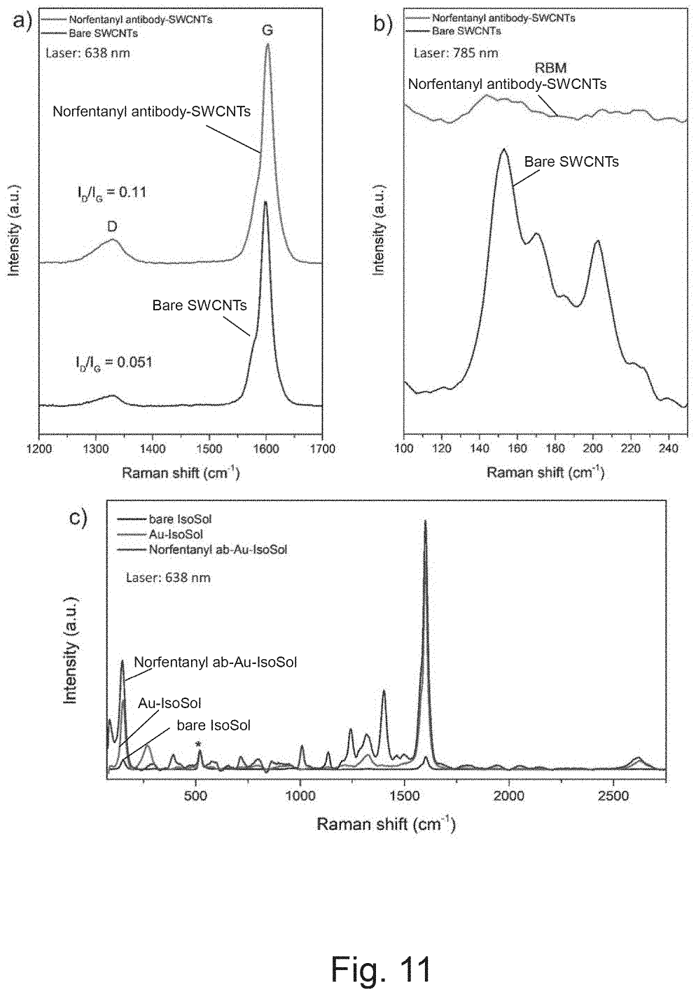

The functionalization of the two types of devices were also characterized by Raman spectroscopy (FIG. 11). For norfentanyl ab-sc-SWCNT devices, the D/G ratio increased from 0.051 to 0.11 after the coupling of norfentanyl antibody, indicating an increase in the degree of functionalization of the carbon nanotubes. Meanwhile, the intensity of RBM peaks decreased significantly after the functionalization of norfentanyl antibody. Such behavior was also observed for BARS-CoV-2 ab functionalized SWCNTs.

For norfentanyl ab-Au-sc-SWCNT devices, a significant increase in the Raman intensity was observed after AuNP deposition due to the surface-enhanced Raman spectroscopy (SERS) effect. More Raman peaks appeared in the range of 500-1500 cm⁻¹ after the addition of norfentanyl antibody. Those peaks might belong to norfentanyl antibody and were enhanced due to the SERS effect.

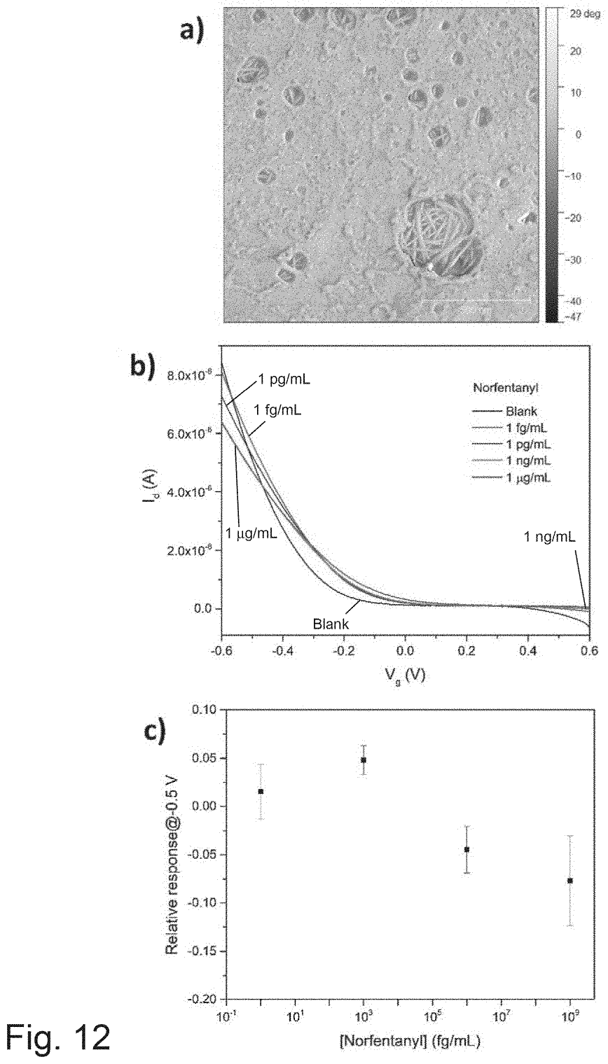

In a number of studied embodiments, fentanyl antibody was used instead of norfentanyl antibody as the norfentanyl receptor on the sc-SWCNTs. Fentanyl antibody has a 13% cross-reactivity with norfentanyl and is more cost-effective. It was observed that the sensing performance was dependent on the amount of antibody functionalized on the se-SWC As shown in FIG. 12a, when fentanyl antibody of 400 μg/mL was attached to the sc-SWCNTs via EDONTIS coupling, the sc-SWCNT surfaces were covered by the antibodies. In this case, the conducting channels were blocked, and the devices showed low sensitivity toward the analyte (FIGS. 12b and 12c).

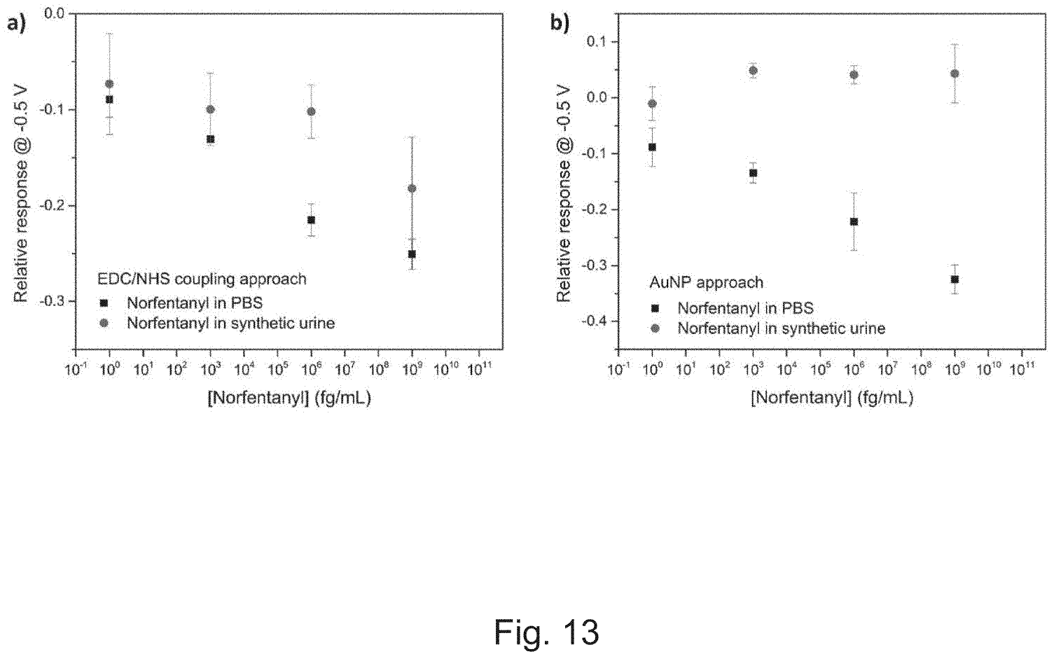

When the antibody concentration was reduced to 100 μg mL, both types of fentanyl antibody-functionalized sc-SWCNT FET sensors demonstrated their sensing capability for norfentanyl (FIGS. 13a and 13b, black data points). However, when testing in synthetic urine, both sensors lost their sensitivity, which was likely a result of the low cross-reactivity/specificity between the fentanyl antibody and norfentanyl. Therefore, norfentanyl antibody was utilized to fabricate the sc-SWCNT F ET sensors in subsequent described below to take advantage of both high sensitivity and specificity of the norfentanyl sensor.

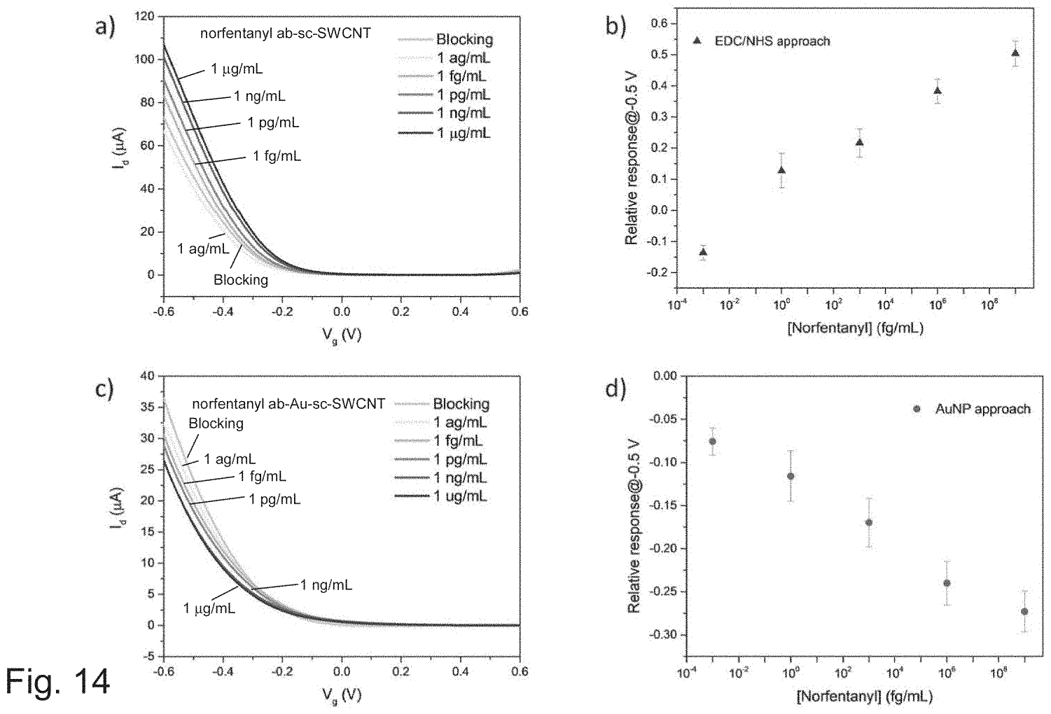

To demonstrate the norfentanyl sensing capabilities of sensors hereof including norfentanyl antibody, both types of devices were exposed to a series of norfentanyl solutions prepared in PBS with concentrations ranging from 1 attogram/mL or ag/mL to 1 μg/mL. In general, the observed results were similar to the results observed for cortisol sensing described above (FIG. 14). The FET sensor employing the direct coupling approach showed an increasing trend when plotting the relative response against the concentration of norfentanyl (in logarithmic scale). While for norfentanyl ab-Au-sc-SWCNT devices, there was a decreasing trend for the calibration curve.

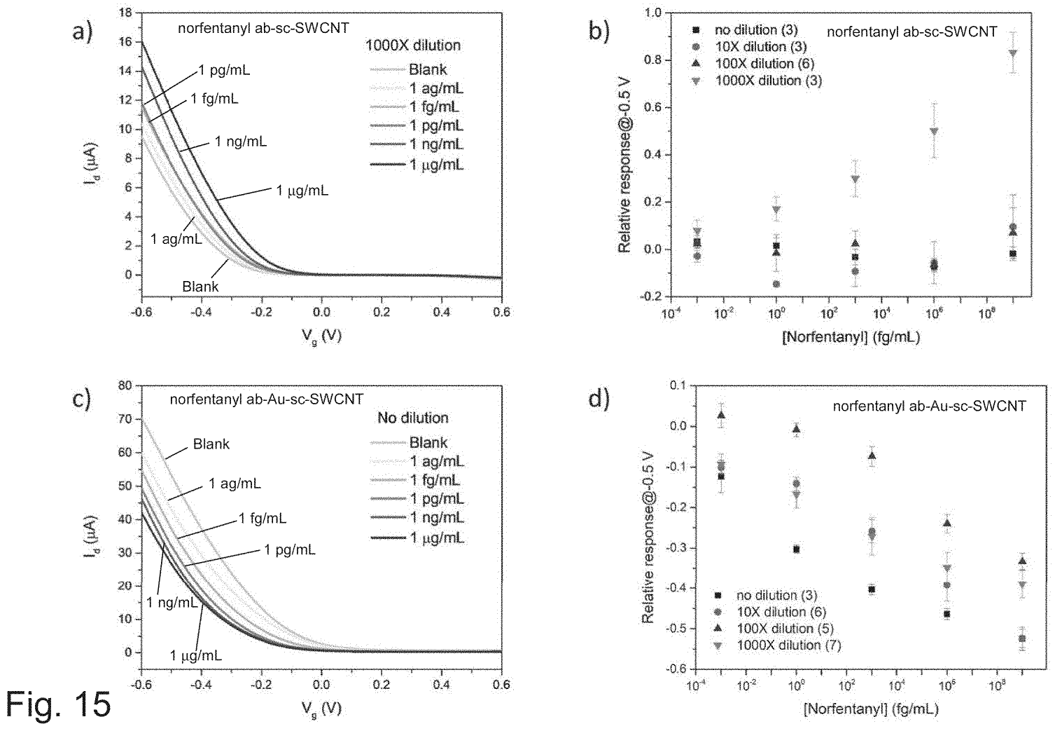

The two types of norfentanyl sensor were also studied in detecting norfentanyl in a more complex system, such as in synthetic urine. To study the possible inference with the interaction between norfentanyl and its antibody arising from other components in the synthetic urine, the devices were tested in different dilutions of synthetic urine. As shown in FIG. 15, when comparing EDC/NHS coupling and AuNP approach, the AuNP decorated biosensors are less susceptible to nonspecific species present in synthetic urine. While the calibration sensitivity was not calculated, the limit of detection is around 1 fg/mL in the case of norfentalyl sensors hereof.

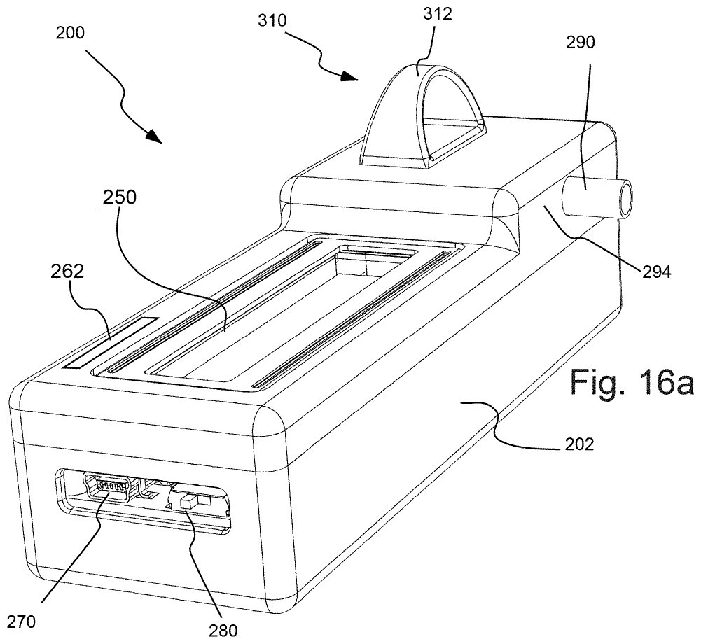

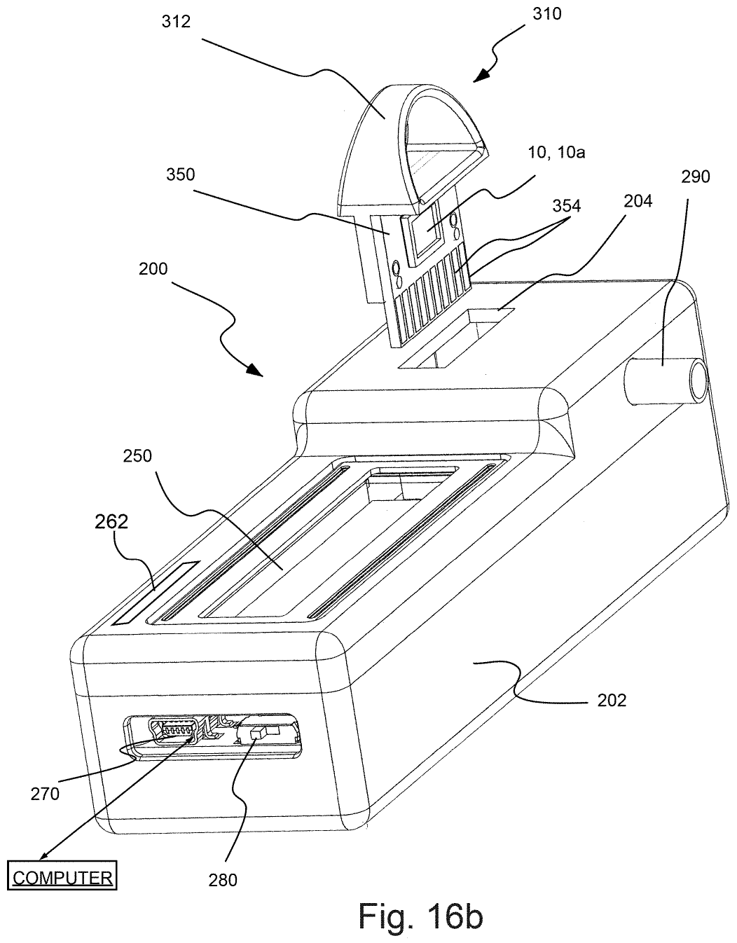

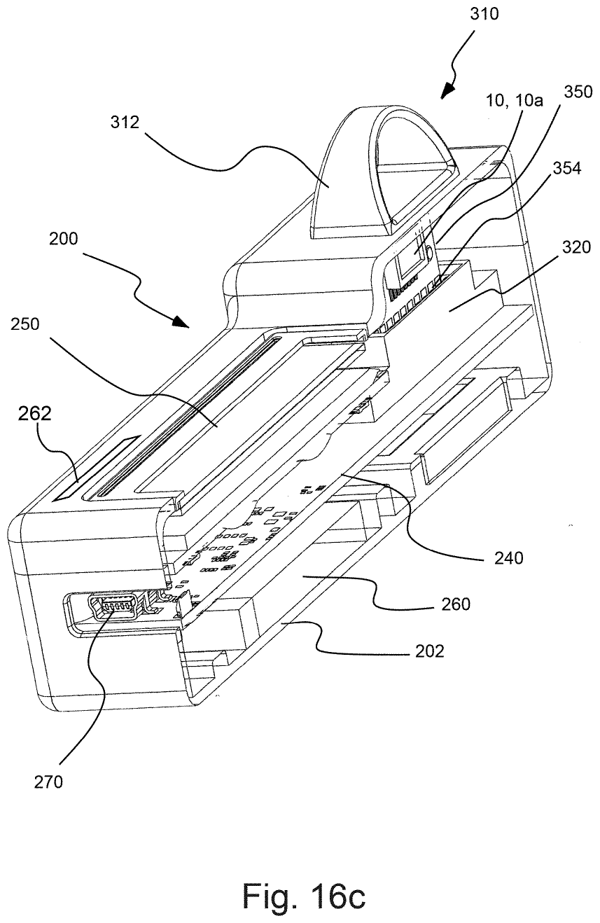

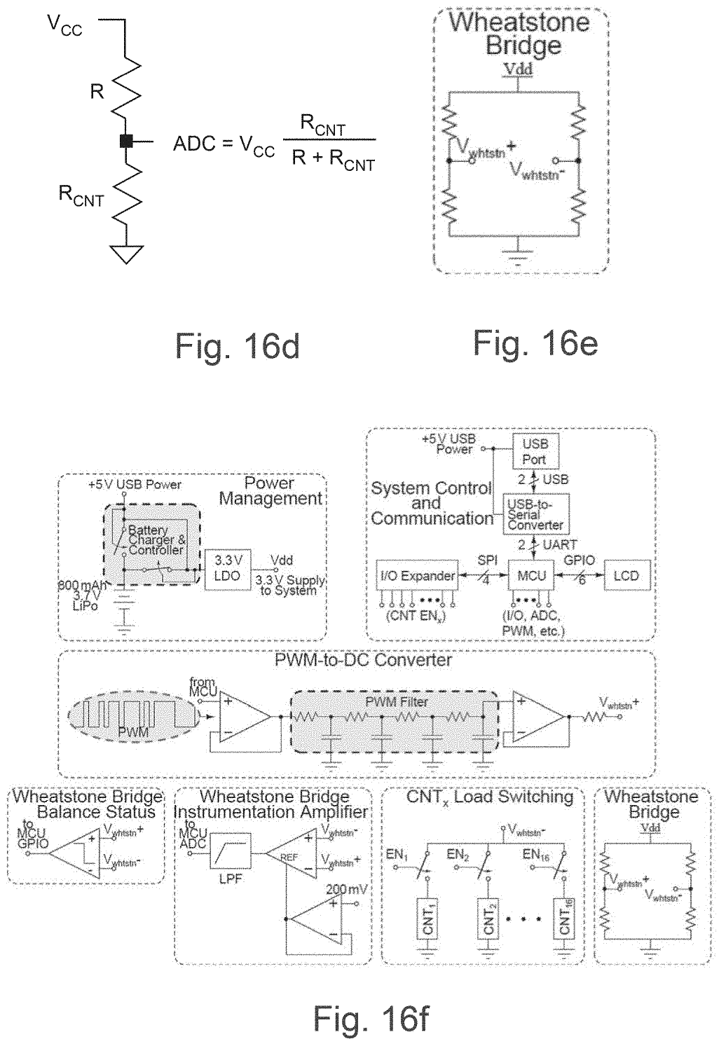

A sensor hereof may be incorporated as the sensing element of handheld sensor device or system 200 (as illustrated in FIGS. 16a through 16b). Such a system may, for example, include a processor system including a CC1110 chip (a system-on-chip including a microcontroller unit or MCC; available from Texas Instruments of Dallas, Tex.) to function as the microcontroller, a 3D printed case or housing (FIGS. 16a through 16c) to house the electronics (FIGS. 16d through 16f). The electronics may include, for example, a Wheatstone Bridge (FIGS. 16e and 16f) to measure the resistance of the sensor. A swappable and insertable sensor board (see, for example. FIGS. 16b and 16c) may, for example, be plugged in and removed from the system.

In the embodiment illustrated in FIG. 16a through 16c, device or system 200 hereof for detection of an antigen or an antibody provides a relatively compact form. Similar devices or systems are, for example, disclosed in PCT International Patent Application No. PCT/US2020/059591 and in U.S. Patent Publication No. 2020/009342, the disclosures of which are incorporated herein by reference. Device 200 includes a microchip-based, sensor assembly 310 in which one or more chemiresistors 10 or field effect transistors (FETs) 10a is/are deposited on a silicon wafer 350 as illustrated in FIGS. 16b and 16c. Sensor assembly 310 is readily removably and operably attachable to a sensor assembly connector or receptacle 320 within housing 202 via a handle or gripping portion 312 on a first or outer end and conductive connectors 354 on a second or inner end. Sensor assembly 310 is placed in connection with a control board 240 (for example, a printed circuit board or PCB) via connector 220.

In the illustrated embodiment, sensor assembly 310 may be placed in and out of connection with connector or receptacle 320 via a slot or opening 204 formed in housing 202. The design of device 200 thereby facilitates removal of sensor assembly 310 for maintenance replacement. A user may, for example, be provided with multiple sensor assemblies 310 in a system of kit for use in connection with device 200. Sensor assemblies 310, when removed from connection with device 200, may, for example, be serviced/refurbished or discarded.

Control board 240 of the electronic circuitry of device 200 includes or has attached thereto a controller system or processor system (not shown, including for example, one or more microprocessors such as a CC1110 microprocessor) and a memory system (not shown) which is placed in operative or communication connection with the processor system via control board 240 or integrated with the processor system. Control board 240 may also be in operative connection with a display 250 such as a liquid crystal display. A power supply/battery 260 (for example, a Lithium Polymer or LiPo battery) may be supplied to power one or more electronic circuitry components as described above. Such electric circuitry components are housed within a housing 202.

A mini USB or other communication port 270 in operative connection with control hoard 240 may extend through housing 202. Mini USB or other communication port 270 may, for example, be used to connect to a. computer such as a general-purpose personal computer or PC (see, for example. FIG. 16b) to, for example, effect software revision and nor data transfer, to effect battery charging and/or to effect power the device (for example, even if battery 260 is absent or damaged) as known in the computer arts. An indicator 262 (for example, one or more LED lights) may be provided to set forth information such as battery status. Status indicator(s) 262 may, for example, indicate when battery 260 is low (RED), when the device is charging battery 260 (BLUE), and when charging of battery 260 is complete (GREEN). An on off or power switch 280 may, for example, be provided on housing 202. A sample port or tube 290 passes through housing 202 and has an outlet in the vicinity of chemiresistor 10a or FET 10 of sensor assembly 210.

Methodologies/circuits for sensing changes in sensor resistance in device or system 200 are shown in FIGS. 16d through 16f. FIG. 16d illustrates a simple voltage divider configuration of resistors, where, a change in resistance is converted to a change in voltage. In the voltage divider network, one resistor (R) is a fixed value, and the other resistance (RCNT) is variable, wherein RCNT represents the sensor resistance. In a number of embodiments, an analog-to-digital converter (ADC) is the input port of a digitizing device, such as a microcontroller/microprocessor. Sensing changes in RCNT is easier when the resistance change results in a larger voltage change. The point where the largest voltage change occurs will be when R equals the nominal sensor resistance before a measurement is taken (for example, R=RCNT).

In a number of embodiments, the resistor network in device 200 is a Wheatstone bridge as illustrated in FIG. 16e, which uses the same principle of voltage division described above but increases the resistance-sensing accuracy with a more complex resistor configuration as illustrated in FIG. 16d. Three of the resistor values are known. The fourth resistor value can be calculated from a measurement of the differential voltage between the centers of each “leg” of the bridge, labeled in FIG. 16e as Vwhtstn. Sensor 10, 10a forms the bottom half of one leg of the bridge. FIG. 16f illustrates an embodiment of electronic circuitry for system 200.

Experimental Section

Materials. Semiconductor-enriched SWCNTs were acquired from Raymor industry, Inc. Polydimethylsiloxane (PDMS) was purchased from Ellsworth Adhesives. HAuCl₄ (99.9%) was purchased from Sigma Aldrich. Chemical compounds that were utilized to prepare an artificial sweat aqueous solution (KCl, NaCl, CaCl₂, MgSO₄, NH₄Cl, uric acid, glucose, lactic acid, and urea) were purchased from Sigma Aldrich.

Cortisol antibody was purchased from ThermoFisher Scientific. (Catalog # PA1-85347). Norfentanyl antibody was purchased from MyBioSource, Inc. (Catalog # MBS5302609). All reagents were analytical grade and used as received. Nanopure water from Thermo Scientific Barnstead Nanopure System with resistivity >18.2 MΩ·cm was used to prepare all solutions.

Device Fabrication

SARS-CoV-2 antibody system. Interdigitated gold electrodes were patterned on a Si/SiO₂ substrate using photolithography, forming 10 μm channels. Semiconducting SWCNTs (IsoSol-S100, Raymor Industries Inc.) were prepared at 0.02 mg/mL in toluene and deposited between gold electrodes via dielectrophoresis (DEP) with an ac frequency of 100 kHz, applied bias voltage of 10 V. and bias duration of 120 s. The devices were annealed at 200° C. for 1 h before use.

The functionalization of the SARS-CoV-2 antibody on SWCNTs was achieved via 1-ethyl-3-(3-dimethylaminopropyl)carbodiimide (EDC/N-hydroxysulfosuccinimide (sulfo-NHS) coupling. Specifically, 50 μL of EDC/sulfo-MiS solution [50 mM/50 mM in 1× phosphate-buffered saline (PBS), pH=5.5] was first added to the devices to activate the carboxylic acid groups on SWCNTs. The devices were then rinsed with nanopure water and incubated with 2 μL of 100 μg/mL SARS/SARS-CoV-2 coronavirus spike protein (subunit 1) polyclonal antibody (Thermo Fisher Scientific, Waltham, Mass., USA: Cat# PA5-817951 for 12 h at 4′C for SAb-functionalized devices and 4 μL of 50 anti-SA RS-CoV-2 NP antibody (Clone#6110) (BioVision Inc., Milpitas, Calif., USA; Cat# A2060) for NAb-functionalized devices. After a thorough rinse with nanopure water, the devices were soaked in a blocking buffer (0.1% Tween 20 and 4% polyethylene glycol in PBS) for 30 min to block unreacted surfaces. After blocking, the devices were rinsed again with nanopure water before any FET measurements.

Norfentanyl and cortisol systems. Interdigitated electrodes (IDEs) on the silicon chips were prepared by standard photolithography process described previously.¹² Afterwards, each chip (2×2 mm) was wire-bonded into standard 40-pin ceramic dual in-line package (CerDIP). Electrical contacts were secured by PDMS, Sc-SWCNTs (5 μL, 0.02 mg/mL in toluene): were deposited onto the surface of the silicon chip by using AC dielectrophoretic (DEP) deposition at 10 Vₚₚand 100 KHz for 120 s. The antibody was then immobilized on the sc-SWCNTs via two different approaches.

EDC/NHS coupling approach: The sc-SWCNT devices were first incubated in a 50 mM/50 mM 1-ethyl-3-(3-dimethyaminopropyl)carbodiimide (EDC)/N-hydroxysulfosuccinimide (sulfo-NHS) solution tin IS phosphate buffered saline, PBS) for 30 min to activate the carboxylic acid groups. For cortisol sensing, cortisol antibody (10 μL, 100 μg/mL in PBS buffer) was then introduced on the sc-SWCNTs surface directly after activation and incubated overnight at 4° C. For norfentanyl sensing, norfentanyl antibody (10 μL, 103.6 μg/mL in PBS buffer) was then introduced on the sc-SWCNTs surface directly after activation and incubated overnight at 4° C.

Gold nanoparticle (AuNP) approach: Gold nanoparticles were deposited on sc-SWCNTs via bulk electrolysis using, a CH Instruments electrochemical analyzer in a three-electrode setup (1 M Ag/AgCl reference electrode, Pt counter electrode, and IDEs as working electrodes) from a AuCk solution (1 mM in 0.1 M HCl). Size of metal nanoparticles was optimized for these experiments through control of deposition voltage (−0.2 V) and time (30 s). For cortisol sensing, cortisol antibody (10 μL, 100 μg/mL in PBS buffer) was then added on the device surface and incubated overnight at 4° C. For norfentanyl sensing, norfentanyl antibody (10 μL, 103.6 μg/mL in PBS buffer) was then added on the device surface and incubated overnight at 4° C.

After the attachment of antibodies on sc-SWCNTs, a blocking buffer was applied to block the unreacted surfaces on the sc-SWCNTs to prevent non-specific binding. The blocking buffer consists of 0.2% Tween 20 and 4% polyethylene glycol (PEG) in PBS. To apply the blocking buffer, 100 μL of the blocking buffer was added to the sensor and incubated for 30 min at room temperature. After the incubation, the blocking buffer was washed off using nanopure water.

Fluorescence Imaging. Fluorescence images were obtained using an Olympus 1X81/1X2-UCB microscope. The enhanced green fluorescent protein (EGFP) antibody (Antibodies-online Inc, Limerick Pa., USA) was immobilized on the SWCNT FET device using the same method as described in the “Device Fabrication” section. EGFP protein solution (2 μL, 10 μg/mL) was then added to the device and incubated for 10 min at room temperature. Fluorescence images before and after the addition of EGFP were captured under an excitation of 489 nm. As a control, 2 μL of 10 μg/mL EGFP protein solution was also added to a bare SWCNT FET device with blocking. Fluorescence images of the bare SWCNT FET device before and after EGFP binding were also taken under an excitation of 489 nm.

Fluorescence Imaging. Fluorescence images were obtained using an Olympus 1X81/1X2-UCB microscope. The enhanced green fluorescent protein (EGFP) antibody (Antibodies-online Inc, Limerick, Pa., USA) was immobilized on the SWCNT FET device using the same method as described in the “Device Fabrication” section. EGFP protein solution (2 μL, 10 μg/mL) was then added to the device and incubated for 10 min at room temperature. Fluorescence images before and after the addition of EGFP were captured under an excitation of 489 nm. As a control, 2 μL of 10 μg/mL EGFP protein solution was also added to a bare SWCNT FET device with blocking. Fluorescence images of the bare SWCNT FET device before and after EGFP binding were also taken under an excitation of 489 nm.

UV-Vis-NIR Absorption Spectroscopy. sc-SWCNTs (100 μL, 0.02 mg/mL) were drop-casted on a 1″×1′ quartz slide and heated at 200 to evaporate the solvent. UV-vis-NIR spectra of the sc-SWCNT were collected using a Perkin Elmer LAMBDA 900 UV-vis-NIR spectrophotometer,

Atomic Force Microscopy. Atomic force microscopy (AFM) data were collected using a Balker Multimode 8 AFM system with a Veeco Nanoscope. IIIa controller in the tapping mode. The AFM image and height profiles were processed and obtained in Gywddion.

X-ray Photoelectron Spectroscopy. X-ray photoelectron spectroscopy (NPS) data were generated on a Thermo ESCALAB 250 Xi APS using monochromated A1 Kα X-rays as the source. A 650 μm spot size was used, and the samples were charge-compensated using an electron flood gun.

Raman Spectroscopy. The XplorA Raman-AEWIERS system was used to record all Raman spectra, The radial breathing mode (RBM) region was recorded using a 785 nm (100 mW) excitation laser operating at 1 power. 1) and G peak regions were recorded using a 638 nm (24 mW) excitation laser operating at 1% lower.

FET Measurements. SARS-CoV-2 antigens. Liquid-gated PET device configuration was employed to study the FET transfer characteristics of SARS-CoV-2 antibody-functionalized FET devices for the detection of SARS-CoV-2 antigens. Nanopure water was used as the gating electrolyte. FET characteristic curves were recorded by collecting the source-drain current (I_{d}), while sweeping the gate voltage from +40.6 to −0.6 V versus a 1 M AglAgCl reference electrode with a fixed drain voltage of 50 mV.

A series of SARS-CoV-2 spike S1-His recombinant protein (Sino Biological, Beijing, China; Cat#40591-V08H) solutions ranging from 0.55 fg/mL to 55 μg/mL and recombinant coronavirus nucleoprotein (BioVision Inc.; Cat# P1523) ranging from 0.016 fg/mL to 16 μg/mL were prepared in PBS. All protein solutions were tested from the lowest to the highest concentrations. For each measurement, 2 μL of the protein solution was added to the antibody-functionalized devices and incubated for 2 min. The devices were then washed three times with nanopure water to remove any unbound protein and measured in nanopure water as the gating electrolyte. After a period of time to allow binding, removal of unbound analyte was found to facilitate achievement of a lower level of detection.

The relative response (R) of each FET device was calculated using R=ΔI/I₀ at −0.5 V_{g}, where ΔI=I_{d}−I₀ and I₀ is the drain current in nanopure water before antigen exposure at −0.5 V_{g}. The final results reported were averaged relative responses of two to six devices with standard deviation (SD) as error bars. The number of devices (n) tested for each experiment was specified in the figures.

NTFET measurements and analysis. Norfentanyl and cortisol antigens. NTFET transfer characteristics were collected using a Keithley 2602b Source Meter. A Ag/AgCl reference electrode (CH11111, CH Instruments, Inc) was used as the liquid gate electrode. Gate voltage (V_{G}) was swept from +0.6 V to −0.6 V while source-drain voltage (Vsn) was held constant at 50 mV. Source drain current was measured as a function of gate voltage V_{G}, Source-drain current (I_{SD}) or conductance (G, calculated as I_{SD}/V_{SD}) was plotted as a function of the applied V_{G}. The NTFET transfer characteristics (I_{SD}-V_{G}or G-V_{G}) were first collected from each device in nanopure water, to be used as a baseline. Afterwards, the analyte solutions were added to the devices, and the solution was incubated with antibody for 10 minutes. After the incubation, the analyte solutions were rinsed off using nanopure water and the NTFET transfer characteristics were measured in nanopure water.

The normalized conductance of each FET device was calculated using (G G₀)/G₀ at a chosen gate voltage, where G₀ is the conductance of the device tested in blank ([Cortisol]=0 M) at the chosen voltage. Due to the sensor-to-sensor variation, the gate voltage chosen for plotting the calibration curve might be different for each sensor to obtain the best calibration. The final results reported in the figures were averaged relative conductance of all devices tested on one sensor chip with standard deviation (SD) as error bars.

Clinical Sample Tests or SARS-CoV-2. Remnant nasopharyngeal swab samples tested by NAAT for SARS-CoV-2 RNA on emergency use authorized platforms for standard clinical care were used under the auspices of the University of Pittsburgh IRB study number 20003220. A total of 28 PCR-positive samples and 10 negative nasopharyngeal swab samples were tested. Each sample (10 μL) was added to the antibody-functionalized devices and incubated for 2 min. After 2 min, the sample was removed from the devices and the devices were washed three times with water. FET measurements were taken in water as the gating electrolyte.

The relative responses were calculated using the same method as described above in in “FET Measurements”. The final results reported were averaged relative responses of one to four devices with SD as error bars. Multiple devices (n) were tested for each sample.

The foregoing description and accompanying drawings set forth a number of representative embodiments at the present time. Various modifications, additions and alternative designs will, of course, become apparent to those skilled in the art in light of the foregoing teachings without departing from the scope, hereof, which is indicated by the following claims rather than by the foregoing description. All changes and variations that fall within the meaning and range of equivalency of the claims are to be embraced within their scope.

What is claimed is:

1. A method of detecting an analyte which is an antigen or an antibody in a fluid sample, comprising:
providing a sensor device including a sensor including a substrate and a sensor medium on the substrate, the sensor medium including a plurality of nanostructures having an enriched semiconducting content and one or more of at least one agent selected from the group consisting of an antibody, an antigen receptor or an antigen immobilized upon at least a portion of the plurality of nanostructures, and electronic circuitry including at least one measurement system in operative connection with the sensor to measure a variable providing a measure of change in at least one property of the sensor medium which is dependent upon the presence of the analyte, wherein the at least one agent is an antibody or an antigen receptor if the analyte is an antigen and the at least one agent is an antigen if the analyte is an antibody;
exposing the sensor the fluid sample far a period of time;
subsequent to exposing the sensor to the fluid sample, washing the sensor one or more times with a liquid of known ionic strength; and
after washing the sensor. measuring an output of the sensor with the liquid of known ionic strength over the sensor medium

2. The method of claim 1 wherein the ionic strength of the liquid is chosen to be less than that of the fluid sample to increase sensitivity compared to an output measured in the presence of the fluid sample,

3. The method of claim 2 wherein the liquid is a purified water.

4. The method of claim 3 wherein the purified water has a resistivity greater than or equal to 18.2 MΩ·cm.

5. The method of claim 2 wherein the one or more of at least one agent selected from the group consisting of an antibody, an antigen receptor or an antigen is covalently attached to at least a portion of the plurality of nanostructures or is attached to at least a portion of a plurality of gold nanoparticles immobilized upon the plurality of nanostructures.

6. The method of claim 2 wherein the plurality of nanostructures has an enriched semiconductine. content of at least 90%.

7. The method of claim 6 wherein the plurality of nanostructures comprises a plurality of carbon nanostructures.

8. The method of claim 7 wherein the plurality of carbon nanostructures comprises single-walled carbon nanotubes.

9. The method of claim 8 wherein the variable measured is an electrical property change.

10. The method of claim 9 wherein the at least one agent is an antibody and the analyte is an antigen.

11. The method of claim 10 wherein the antigen is an antigen of a pathogen selected from the group of a viral pathogen and a bacterial pathogen.

12. The method of claim 11 wherein the pathogen is SARS-CoV-2, HIV, tuberculosis, syphilis, hepatitis, E. coli, Salmonella, Pseudomonas aeruginosa, Influenza, Staphylococcus aureus, or Streptococcus pyogenes, cytomegalovirus, Epstein-Barr virus (EBV), or an autoimmune pathogen.

13. The method of claim 11 wherein the pathogen is SAPS-CoV-2.

14. The method of claim 13 wherein the antigen is a spike antigen (SAg) or a nucleocapsid protein antigen (NAg) of SARS-CoV-2 and the antibody is anti-SARS-CoV-2 spike protein antibody (SAb) or anti-SARS-CoV-2 nucleocapsid protein antibody.

15. The method of claim 14 wherein the antigen is a spike antigen (SAg of SARS-CoV-2 and the antibody in the anti-SARS-CoV-2 spike protein antibody.

16. The method of claim 10 wherein the antigen is a hormone or an opioid.

17. The method of claim 16 wherein the antigen is cortisol or norfentanyl.

18. The method of claim 1 wherein the sensor is incorporated within a field effect transistor circuit of the electronic circuitry and the liquid functions as a liquid gate.

19. The method of claim 1 wherein the sensor device includes a plurality of the Sensors.

20. The method of claim 19 wherein the sensor media of one or more of the plurality of sensors includes one or more of a first agent selected from the group consisting of an antibody, an antigen receptor or an antigen covalently attached to at least a portion of the plurality of nanostructures and the sensor media of one or more others of the plurality of sensors includes one or more of a second agent selected from the group consisting of an antibody, an antigen receptor or an antigen covalently attached to at least a portion of the plurality of nanostructures, wherein the first agent is different from the second agent.

21. The method of claim 1 wherein the sensor includes more than one agent selected from the group consisting of an antibody, an antigen receptor or an antigen covalently attached to at least a portion of the plurality of nanostructures.

22. The method of claim 1 wherein the at least one agent is maintained in a liquid phase.

23. A method of detecting an analyte which is an antigen or an antibody in a fluid sample, comprising:
exposing a sensor including a substrate and a sensor medium on the substrate to the fluid sample for a period of time, the sensor medium including a plurality of nanostructures and one or more of at least one agent selected front the group consisting of an antibody, an antigen receptor or an antigen immobilized upon at least a portion of the plurality of nanostructures, wherein the at least one agent is an antibody or an antigen receptor if the analyte is an antigen and the at least one agent is an antigen if the analyte is an antibody,
subsequent to exposing the sensor to the fluid sample, washing the sensor one or more times with a liquid of known ionic strength; and
after washing the sensor, measuring an output of the sensor with the liquid of known ionic strength over the sensor medium.

24. The method of claim 23 wherein the ionic strength of the liquid is chosen to be less than that of the fluid sample to increase sensitivity compared to output measured in the presence of the fluid sample.

25. The method of claim 24 wherein the liquid is a purified water.

26. The method of claim 25 wherein the purified water has a resistivity greater than 18.2 MΩ·cm.
